(19) United States
(12) Patent Application Publication
(10) Pub. No.: US 2004/0019026 A1
(43) Pub. Date: Jan. 29, 2004

(54) Compounds useful for treating hypertriglyceridemia

(76) Inventor: Arthur G. Schwartz, Perkasie, PA (US)

Correspondence Address: SCULLY SCOTT MURPHY & PRESSER, PC, 400 GARDEN CITY PLAZA, GARDEN CITY, NY, 11530

(21) Appl. No.: 10/408,466

(22) Filed: Apr. 7, 2003

Related U.S. Application Data
(63) Continuation-in-part of application No. PCT/US01/31568, filed on Oct. 9, 2001.
(60) Provisional application No. 60/238,659, filed on Oct. 6, 2000.

(51) Int. Cl. A61K 31/57; A61K 31/56

(52) U.S. Cl. 514/177; 514/178

(57) ABSTRACT

The present invention is directed to a method for treating a patient having hypertriglyceridemia comprising administering thereto a compound of the formula:

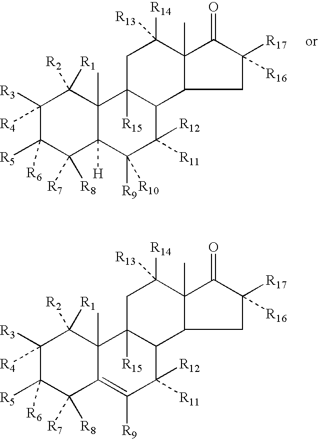

### CROSS REFERENCE TO RELATED APPLICATION

This application is a continuation in part of international application PCT/US 01/31568 filed on Oct. 9, 2001 and is claiming priority of U.S. Provisional Application No. 60/238,659 filed on Oct. 6, 2000.

### FIELD OF THE INVENTION

The present invention relates to the use of derivatives of 5-androsten-17-one and 5-androstan-17-one for lowering triglycerides in patients who have hypertriglyceridemia, especially those having low levels of HDL cholesterol, and/or those who are obese.

### BACKGROUND OF THE INVENTION

Hyperlipidemia, a condition, which is characterized by an abnormal increase in serum lipids, i.e., cholesterol, triglycerides and phospholipids, is a primary cause for cardiovascular disease (CVD) and other peripheral vascular diseases. Hyperlipidemics having high levels of LDL (Low Density Lypoprotein) and VLDL (Very Low Density Lipoprotein) cholesterol are at risk for CVD.

One form of hyperlipidemia is hypertriglyceridemia, a condition in which there is an excessive amount of triglycerides in the plasma. It is a common lipid abnormality afflicting about 20% of the middle-aged human population in the U.S. A patient suffering from hypertriglyceridemia is at risk for atherosclerosis and CHD. Moreover, hypertriglyceridemia in combination with low levels of plasma HDL cholesterol (high density lipoprotein cholesterol, sometimes designated as the good cholesterol) is associated with insulin resistance, and both independently are risk factors for coronary heart disease and other peripheral vascular diseases. In fact, the major lipid abnormality in Type II diabetes is hypertriglyceridemia.

Insulin resistance is a disorder of glucose metabolism. Patients with insulin resistance have a diminished ability to properly utilize glucose. In insulin resistance, there is a diminished ability of insulin to exert its biological action. The body secretes abnormally high amounts of insulin to compensate for this defect, failing which; the plasma glucose concentration inevitably rises. Insulin resistance can cause or contribute to hypertension, obesity, atherosclerosis and a variety of other disorders. Eventually, it can progress to a point where a diabetic state is reached. Insulin resistant (or Type II) diabetes is a severe and potentially disabling disease, if not properly treated.

Insulin resistance and hypertriglyceridemia both have a contributory role in obesity, cardiovascular disease, atherosclerosis and Type II diabetes mellitus.

Thus, therapeutic agents, which improve insulin resistance, lower plasma triglycerides, and increase HDL will have great significance in preventing cardiovascular morbidity and improving quality of life.

General measures such as weight reduction, exercise and avoidance of alcohol are initially used to control hypertriglyceridemia. If, however, triglyceride levels remain high, treatment with a fibric acid derivative, such as gemfibrozil or nicotinic acid, is frequently used. Gemfibrozil lowers triglycerides about 25-35%, and modestly raises HDL levels, but has no effect on insulin resistance. Nicotinic acid worsens insulin resistance. Moreover, nicotinic acid causes numerous side effects, including intense flushing and associated pruritus, which limits its use.

Thus, the search continues for effective drugs, which are capable of treating hypertriglyceridemia, raising HDL levels and treating insulin resistance. The present inventor has found such drugs. The drugs are derivatives of 5-androsten-17-ones and 5α-androstan-17-ones.

U.S. Pat. Nos. 5,804,576 and 5,714,481 describe 5-androsten-17-ones having the formula:

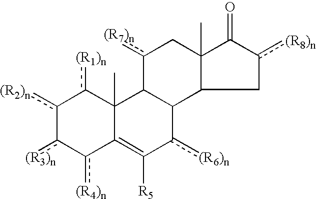

wherein R₁, R₂, R₃, R₄, R₆, R₇ and R₈ are each independently selected from the group consisting of hydrogen, alkyl, alkenyl, alkynyl, halogen and hydroxyl; R₅ is hydrogen, alkyl, alkenyl, alkynyl or halogen, n is an integer from 1 to 2 inclusive with the proviso that when R₁, R₂, R₃, R₄, R₅, R₆, R₇ or R₈ is alkenyl or alkynyl, n is 1; and with further provisos that at least one of R₁, R₂, R₃, R₄, R₅, R₆, R₇ or R₈ is other than hydrogen; that when R₃ is hydroxy, any one of the substituents R₂, R₄, R₅, R₆, R₇ or R₈ is other than hydrogen and R₁ is other than hydrogen or hydroxy; when R₃ is hydroxy, R₁ may only be alkyl when any one of R₂, R₄, R₅, R₆, R₇ or R₈ is other than hydrogen; when R₃ is hydroxy, R₄ may only be halogen or hydroxy when R₁, R₂, R₅, R₆, R₇ or R₈ is other than hydrogen; when R₃ is hydroxy, R₆ may only be hydroxy when R₁, R₂, R₄, R₅, R₇ or R₈ is other than hydrogen; when R₃ is hydroxy, R₂ may only be alkyl when one of R₁, R₄, R₅, R₆, R₇ or R₈ is other than hydrogen; when R₃ is hydroxy, R₆ can only be methyl when R₁, R₂, R₄, R₇ or R₈ is other than hydrogen and R₅ is other than hydrogen or methyl; when R₃ is hydroxy, R₇ may only be hydroxy when R₁, R₂, R₄, R₅, R₆ or R₈ is other than hydrogen; when R₃ is hydroxy, R₈ may only be methyl, ethyl, isopropyl, hydroxy or halogen when R₁, R₂, R₄, R₅, R₆ or R₇ is other than hydrogen; when R₃ is hydroxy, R₅ may only be alkyl when R₁, R₂, R₄ or R₇ is other than hydrogen and R₆ or R₈ is other than hydrogen or methyl; when R₃ is fluorine, any one of the substituents R₁, R₂, R₄, R₅, R₆, R₇ or R₈ is other than hydrogen; when R₃ is iodine or chlorine, R₅ may only be methyl when R₁, R₂, R₄, R₆, R₇ or R₈ is other than hydrogen; and when R₃ is hydroxy, R₄ may only be hydroxy when R₁, R₂, R₅, R₆ or R₈ is other than hydrogen. They also disclose 16α-fluoro-5α-androstan-17-ones of the formula:

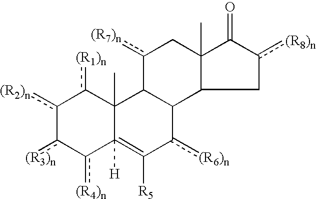

wherein R₁, R₂, R₃, R₄, R₆, R₇ or R₈ are selected from the group consisting of hydrogen, alkyl, alkenyl, alkynyl, halogen and hydroxyl, R₅ is hydrogen, alkyl, alkenyl, hydroxy, alkynyl or halogen, n is an integer from 1 to 2 inclusive with the proviso that when R₁-R₈ are alkenyl or alkynyl, then n is 1 and with the further provisos that R₃ may be hydroxy or halogen only when any one of R₁, R₂, R₄, R₅, R₆, R₇ or R₈ is other than hydrogen; when R₃ is hydroxy, R₁ may be hydroxy or halogen only when any one of R₂, R₄, R₅, R₆, R₇ or R₈ is other than hydrogen; when R₃ is hydroxy, R₂ may be methyl or halogen only when any one of R₄, R₅, R₆, R₇ or R₈ is other than hydrogen; when R₃ is hydroxy, R₄ may be halogen, methyl or hydroxy only when any one of R₁, R₂, R₃, R₅, R₆, R₇ or R₈ is other than hydrogen; when R₃ is hydroxy, R₅ may be methyl, halogen or hydroxy only when R₁, R₂, R₄, R₆, R₇ or R₈ is other than hydrogen; when R₃ is hydroxy, R₆ may be hydroxy or methyl only when R₁, R₂, R₄, R₅, R₇ or R₈ is other than hydrogen; when R₃ is hydroxy, R₇ may be hydroxy only when R₁, R₂, R₄, R₅, R₆ or R₈ is other than hydrogen; when R₃ is hydroxy, R₈ may be methyl, hydroxy or halogen only when R₁, R₂, R₄, R₅, R₆ or R₇ is other than hydrogen; R₇ may be only hydroxy when anyone of R₁, R₂, R₃, R₄, R₅, R₆ and R₈ is other than hydrogen; and R₈ may be bromo only when R₁, R₂, R₃, R₄, R₅, R₆ or R₇ is other than hydrogen.

They describe that these compounds are useful for treating, inter alia, diabetes and hyperlipidemia.

U.S. Pat. Nos. 5,744,462, 5,700,793, 5,696,106, 5,656,621, and 5,157,031 describe steroids of the formula:

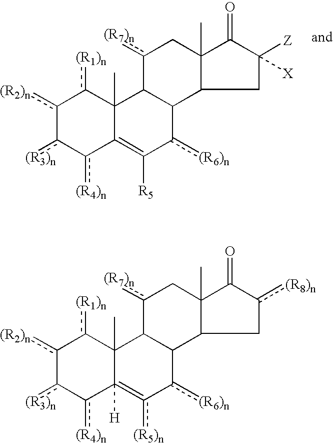

wherein

R₁, R₂, R₄, R₅, R₆ and R₇ are each independently hydrogen or lower alkyl;

R₃ is hydrogen;

X is halogen, hydroxy, hydrogen, lower alkyl, or lower alkoxy;

Z is lower alkyl or hydrogen; and

n is 1 or 2, with the proviso that at least one of X and Z is other than hydrogen.

They teach that these compounds are useful for treating, inter alia, hyperlipidemia and diabetes.

U.S. Pat. No. 5,001,119 disclose compounds of the formula:

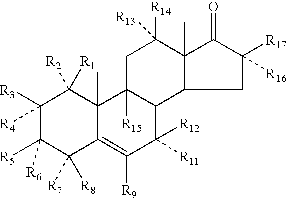

wherein

R₁, R₂, R₃, R₄, R₅, R₆, R₇, R₈, R₁₁, R₁₂, R₁₃, R₁₄ and R₁₅ are independently hydrogen, lower alkyl, halogen, hydroxy or lower alkoxy;

R₉ is hydrogen, lower alkyl or halogen; and

R₁₆ and R₁₇ are independently hydrogen, amino, loweralkylamino, diloweralkylamino, aminoloweralkyl, loweralkyl aminolower alkyl, diloweralkylaminolower alkyl, loweralkoxyloweralkyl, lower alkoxy, hydroxy lower alkyl, monohaloloweralkyl, dihaloloweralkyl, trihaloloweralkyl, loweralkanoyl, formyl, lower carbalkoxy, or lower alkanoyloxy or R₁₆ and R₁₇ taken together with the carbons to which they are attached form a lower cycloalkyl or a cyclic ether containing one ring oxygen atom and up to 5 ring carbon atoms with the proviso that when R₅ is hydroxy and R₁, R₂, R₃, R₄, R₆, R₇, R₈, R₉, R₁₁, R₁₂, R₁₃, R₁₄ and R₁₅ are hydrogen, then R₁₆ is other than CH₂N(CH₃) and with the further proviso that R₁₆ and R₁₇ are not hydrogen simultaneously.

It also discloses compounds of the formula:

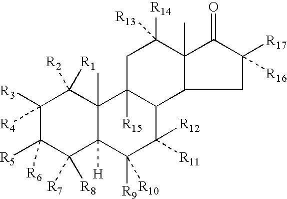

wherein

R₁, R₂, R₃, R₄, R₅, R₆, R₇, R₈, R₁₁, R₁₂, R₁₃, R₁₄ and R₁₅ are independently hydrogen, lower alkyl, halogen, hydroxy or lower alkoxy;

R₉ and R₁₀ are independently loweralkyl, hydrogen or halogen; and

R₁₆ and R₁₇ are independently amino, lower alkylamino, diloweralkyl amino, aminoloweralkyl, loweralkyl aminoloweralkyl, diloweralkylamino loweralkyl, lower alkoxy, hydroxyloweralkyl, monohaloloweralkyl, dihaloloweralkyl trihaloloweralkyl, loweralkoxyloweralkyl, loweralkanoyl, formyl, lower carbalkoxy, hydrogen or lower alkanoyloxy; or

R₁₆ and R₁₇ taken together with the carbon to which they are attached form a lower cycloalkyl or a cyclic ether containing one ring oxygen atom and up to 5 ring carbon atoms, with the further proviso that R₁₆ and R₁₇ are not hydrogen simultaneously.

It discloses that these compounds are useful pharmaceuticals.

However, none of the aforementioned patents teach that the androstene and androstane derivatives described hereinbelow lower plasma triglyceride levels, especially in those patients who suffer from hypertriglyceridemia and who have low HDL levels. Moreover, none of the aforementioned patents teach that the androstene and androstane derivatives described hereinbelow lower triglyceride levels of patients who suffer from hypertriglyceridemia and insulin resistance.

### SUMMARY OF THE INVENTION

Accordingly, the present invention is directed to the method of treating a patient suffering from hypertriglyceridemia comprising administering thereto a therapeutically (or antiglucocorticoid) effective amount of a compound of the formula:

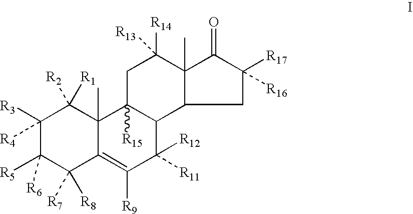

wherein

R₁, R₂, R₃, R₄, R₇, R₈, R₁₁, R₁₂, R₁₃, R₁₄ and R₁₅ are independently hydrogen, alkyl, halogen, hydroxy or alkoxy;

R₅ and R₆ are independently hydrogen, alkyl, alkoxy or halogen or hydroxy;

R₉ is hydrogen, alkyl, halogen or alkoxy;

R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxyloweralkyl, aminoloweralkyl, loweralkylaminoloweralkyl, diloweralkylamino lower alkyl, haloloweralkyl, dihaloloweralkyl or trihaloloweralkyl, with the proviso that only one of R₁₆ and R₁₇ may be alkenyl or alkynyl and with the further proviso that if R₅ or R₆ is hydroxy, then R₁₆ is other than hydrogen.

The present invention is also directed to a method for treating a patient having hypertriglyceridemia, comprising administering thereto a therapeutically (or antiglucocorticoid) effective amount of a compound of the formula:

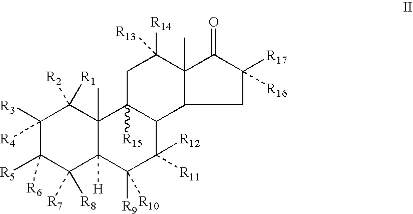

wherein

R₁, R₂, R₃, R₄, R₇, R₈, R₁₁, R₁₂, R₁₃, R₁₄ and R₁₅ are independently hydrogen, alkyl, halogen, hydroxy or alkoxy;

R₅ and R₆ are independently hydrogen, hydroxy alkyl, alkoxy or halogen;

R₉ is hydrogen, alkyl, halogen or alkoxy;

R₁₆ and R₁₇ are independently hydrogen, alkyl, halogen, hydroxy, alkoxy, lower alkenyl, lower alkynyl, amino, lower alkylamino, diloweralkylamino, loweralkoxy lower alkyl, hydroxyloweralkyl, aminoloweralkyl, loweralkylaminoloweralkyl, diloweralkylamino lower alkyl, haloloweralkyl, dihaloloweralkyl or trihaloloweralkyl, with the proviso that only one of R₁₆ and R₁₇ may be alkenyl or alkynyl.

The present invention is also directed to a method of treating a patient having hypertriglyceridemia, said method comprising administering thereto a therapeutically effective amount of a compound of Formula I or Formula II. It is also directed, in another embodiment to treating a patient having hypertriglyceridemia and at least one of the following characteristics (a) insulin resistance; (b) obesity, especially with a BMI>30; (c) low HDL levels, said method comprising administering thereto a therapeutically effective amount of a compound of Formula I or II. The present invention is also directed to reducing the adverse effects of enhanced glucocorticoid activity in a mammal, including humans which comprise administering to said animal an anti-glucocorticoid effective amount of compounds of Formula I or II. The adverse effects may result from various factors, such as hypersecretion of glucocorticoids; the enzymatic action of 11β-hydroxysteriod dehydrogenase which converts cortisone to cortisol; the administration of glucocorticoids to the animal, and the like. These factors may result in enhanced glucocorticoid action which may manifest in certain diseases, symptoms, conditions or malady or side effects, resulting from the administration of glucocorticoids administration. Thus, the compounds of Formula I and II may be used to treat, ameliorate, prevent or retard the progression of an unwanted condition or symptom or malady in a patient relating to the enhanced antiglucocorticoid effect. Alternatively, if glucocorticoids are being administered to the patient e.g. for treatment, the compounds of Formula I or II may be coadministered in antiglucocorticoid effective amount to reduce, prevent the side effects associated with glucocorticoids treatment.

### DETAILED DESCRIPTION OF THE INVENTION

The compounds utilized in the present invention are steroids. In accordance with I.U.P.A.C. nomenclature, the carbon atoms of the present invention are numbered as follows and the steroids have the designated I.U.P.A.C. stereochemistry:

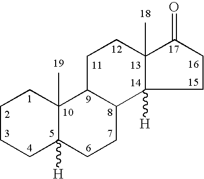

The various substituents are designated as being in the α-position by means of a broken line (---) joining the substituent to the steroid nucleus. The substituents are designated as being in the β-position by means of a solid line (—) joining the substituent to the steroid nucleus. In those cases in which the substituents may be either in the α-or β-position, the substituents are indicated as being joined to the steroid nucleus by a broken line and solid line placed side by side.

As used herein, the term “alkyl”, when used alone or in combination has 1-12 carbon atoms. The term “lower alkyl”, refers to an alkyl group having one to six carbon atoms. The alkyl groups may be straight chain or branched. Examples include methyl, ethyl, propyl, isopropyl, butyl, isobutyl, sec-butyl, t-butyl, pentyl, neopentyl, and hexyl. It is preferred that the alkyl group is lower alkyl. The preferred lower alkyl group contains 1-3 carbon atoms. The most preferred alkyl group is methyl.

The term “alkoxy” when used alone or in combination as used herein, refers to an alkoxy group having 1-12 carbon atoms. As used herein, the term “lower alkoxy”, refers to an alkoxy group having 1-6 carbon atoms. It many be straight chain or branched. Examples include methoxy, ethoxy, propoxy, isopropoxy, butoxy, isobutoxy, pentoxy and the like. It is preferred that alkoxy is lower alkoxy. It is more preferred that alkoxy contains 1-3 carbon atoms. The most preferred alkoxy group is methoxy.

The halo atoms are preferably Br, I and especially Cl and most especially F.

The term “loweralkylamino”, when used alone or in combination, refers to amino group bonded directly to the steroid nucleus and attached to the amino group is one alkyl group, i.e., —NHR₁₀₀ wherein the nitrogen atom is bonded to the steroid nucleus, and R₁₀₀ is lower alkyl, as defined herein. On the other hand, the term “lowerdialkylamino” refers to an amino group bonded directly to the steroid nucleus and attached to the amino group are two lower alkyl groups which may be the same or different, i.e., N(R₁₀₀)(R₁₀₁), wherein R₁₀₀ and R₁₀₁ are lower alkyl, as defined herein.

The term “hydroxyloweralkyl”, as used herein refers to a lower alkyl as defined herein, which is substituted by a hydroxy group. The hydroxy group may be substituted at any position on the alkyl chain.

The term “loweralkoxy loweralkyl” as used herein refers to a lower alkyl group as defined herein which is bonded to the steroid nucleus, which alkyl group is substituted at any position of the alkyl chain with a lower alkoxy group, as defined herein.

The term “amino lower alkyl” as used herein refers to a lower alkyl group, as defined herein, bonded to the steroid nucleus, which alkyl group is substituted by an amino group. The amino group may be substituted in any position of the alkyl chain.

The term “loweralkylaminoloweralkyl” as used herein, refers to a lower alkyl group, as defined herein, bonded to the steroid nucleus, and the lower alkyl group is substituted with a lower alkylamino group as defined herein, e.g., NHR₁₀₂, wherein R₁₀₂ is loweralkyl and wherein the nitrogen atom is bonded to the alkyl substituent which is bonded to the steroid nucleus. The lower alkylamino group may be substituted on any position of the lower alkyl substituent.

The term “lowerdialkylamino loweralkyl,” refers to a loweralkyl group, as defined herein which is substituted with a diloweralkyl amino group, e.g., —NR₁₀₂R₁₀₃ wherein R₁₀₂ and R₁₀₃ are independently lower alkyl and the nitrogen atom is bonded to the alkyl substituents which is bonded directly to the steroid nucleus. The diloweralkylamino group may be substituted on any position of the loweralkyl substituent.

The term “monohaloloweralkyl” refers to a loweralkyl group which is substituted by halo, as defined herein. The halo group may be substituted on any position of the lower alkyl substituent.

The term “dihaloloweralkyl” refers to a lower alkyl group which is substituted by two halo groups. It is preferred that the two halo groups are on the same carbon. It is also preferred that the two halo groups are the same. It is most preferred that the halo groups are chloro and especially fluoro. Examples include difluoromethyl, dichloromethyl, 2,2-difluoroethyl, and the like.

The term “trihaloloweralkyl” refers to a lower alkyl group which is substituted by three halo groups. It is preferred that the halo groups are the same. It is also preferred that the three halo groups are substituted on the same carbon. Examples include trifluoromethyl, tribromomethyl, trichloromethyl, 2,2,2-trifluoroethyl, 1,2,2-trifluoroethyl and the like. The most preferred is trifluoromethyl.

The term “lower alkenyl” as used herein refers to an alkenyl group which contains two to six carbon atoms and at least one double bond. The alkenyl group may be straight chained or branched and may be in either the Z or E form. Examples include ethenyl, 1-propenyl, 2-propenyl, 1-butenyl, 2-butenyl, isopropenyl, isobutenyl, 1-pentenyl, (Z)-2-pentenyl, (E)-2-pentenyl, (Z)-4-methyl-2-pentenyl, (E)-4-methyl-2-pentenyl, pentadienyl, e.g., 1-3 or 2,4-pentadienyl, 1,3-butadienyl and the like. The preferred alkenyl group is ethenyl.

The term “lower alkynyl” refers to an alkynyl group containing 2-6 carbon atoms and at least one carbon-carbon triple bond. The alkynyl group may be straight chained or branched and may be either the E or Z form. Examples include 1-propynyl, 2-propynyl, 1-butynyl, 2-butynyl, 1-pentynyl, 2-pentynyl, 3-methyl-1-pentynyl, 3-pentynyl, 1-hexynyl, 2-hexynyl, 3-hexynyl, and the like. The preferred alkynyl group is ethynyl.

Thus, in some embodiments, the formula I or formula II compound is a compound wherein R₁, R₂, R₃, R₄, R₇, R₈, R₉, R₁₀, R₁₁, R₁₂, R₁₃, R₁₄ and R₁₅ are independently —H, —OH, —F, —Cl, —Br, —I, —OCH₃, —OC₂H₅, —OCH₂CH₂CH₃, —OCH(CH₃)₂, —OCH₂CH₂CH₂CH₃, —OCH₂CH(CH₃)₂, —OCH(CH₃)CH₂CH₃, —OC(CH₃)₃, —OC₅H₁₁, —OC₆H₁₃, —OC₇H₁₅, —OC₈H₁₇, —CH₃, —C₂H₅, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, —C₅H₁₁, —C₆H₁₃, —C₇H₁₅ or —C₈H₁₇; R₅ and R₆ are defined as hereinabove for R₁ except neither R₅ or R₆ are OH, R₁₆ and R₁₇ are defined independently —H, —OH, —NH₂, —F, —Cl, —Br, —I, —OCH₃, —OC₂H₅, —OCH₂CH₂CH₃, —OCH(CH₃)₂, —OCH₂CH₂CH₂CH₃, —OCH₂CH(CH₃)₂, —OCH(CH₃)CH₂CH₃, —OC(CH₃)₃, —OC₅H₁₁, —OC₆H₁₃, —OC₇H₁₅, —OC₈H₁₇, —CH₃, —C₂H₅, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, —C₅H₁₁, —C₆H₁₃, —C₇H₁₅, —C₈H₁₇, —COH, —CHCH₂, —CHCHCH₃, —CHCHCH₂CH₃, —NHCH₃, —NHC₂H₅, —NHCH₂CH₂CH₃, —NHCH(CH₃)₂, —NHCH₂CH₂CH₂CH₃, —NHCH₂CH(CH₃)₂, —NHCH(CH₃)CH₂CH₃, —NHC(CH₃)₃, —NHC₅H₁₁, —NHC₆H₁₃, —NHC₇H₁₅, —NHC₈H₁₇, —CH₂OH, —C₂H₄OH, —C₃H₆OH, —C₄H₈OH, —C₅H₁₀OH, —C₆H₁₂OH, —C₇H₁₄OH, —C₈H₁₆OH, —CH₂NH₂, —C₂H₄NH₂, —C₃H₆NH₂, —C₄H₈NH₂, —C₅H₁₀NH₂, —C₆H₁₂NH₂, —C₇H₁₄NH₂, —C₈H₁₆NH₂, —CH₂NH₂CH₃, —C₂H₄NH₂C₂H₅, —C₃H₆NH₂C₃H_{7, —C}₄H₈NH₂C₄H₉, —C₅H₁₀NHC₅H₁₁, —C₆H₁₂NHC₆H₁₃, —C₇H₁₄NHC₇H₁₅, —C₈H₁₆NHC₈H₁₇, CH₂F, —C₂H₄F, —C₃H₆F, —C₄H₈F, —C₅H₁₀F, —C₆H₁₂F, —C₇H₁₄F, —C₈H₁₆F, —CH₂Cl, —C₂H₄Cl, —C₃H₆Cl, —C₄H₈Cl, —C₅H₁₀Cl, —C₆H₁₂Cl, —C₇H₁₄Cl, —C₈H₁₆Cl, —CH₂Br, —C₂H₄Br, —C₃H₆Br, —C₄H₈Br, —C₅H₁₀Br, —C₆H₁₂Br, —C₇H₁₄Br, —C₈H₁₆Br, —CH₂I, —C₂H₄I, —C₃H6I, —C₄H₈I, —C₅H₁₀I, —C₆H₁₂I, —C₇H₁₄I or —C₈H₁₆I. In some of these embodiments 2, 3, 4, 5, 6, 7, 8, 9, 10, 11, 12, 13 or 14 of the R₁, R₂, R₃, R₄, R₅, R₆, R₇, R₈, R₉, R₁₀, R₁₁, R₁₂, R₁₃, R₁₄ and R₁₅ variable groups are —H and the remaining variable groups are not —H.

It is preferred that R₁, R₂, R₃, R₄, R₇, R₈, R₉, R₁₃, R₁₄ and R₁₅ are independently hydrogen, halogen, (especially chloro and more especially fluoro), hydroxy, alkyl containing 1-3 carbon atoms or alkoxy containing 1-3 carbon atoms, especially methoxy. It is most preferred that R₁, R₂, R₃, R₄, R₇, R₈, R₁₃, R₁₄ and R₁₅ are hydrogen, hydroxy, methyl, and halo (especially chloro and more especially fluoro) or methoxy. It is most preferred that R₁, R₂, R₃, R₄, R₇, R₈, R₉, R₁₃, R₁₄, R₁₅ are hydrogen.

It is preferred that R₁₁ and R₁₂ are independently hydrogen, halogen (especially chloro and more especially fluoro), hydroxy, alkyl containing 1-3 carbon atoms (especially methyl) or alkoxy containing 1-3 carbon atoms, especially methoxy. It is most preferred that one of R₁₁ and R₁₂ is hydrogen and the other is as defined hereinabove. It is most preferred that one of R₁₁ and R₁₂ is hydrogen and the other is hydrogen, or hydroxy or methoxy or fluoro or chloro or methyl.

When the compound utilized is an androstene, it is preferred that R₉ is hydrogen, alkyl containing 1-3 carbon atoms or alkoxy containing 1-3 carbon atoms or halo, especially fluoro or chloro. It is most preferred that R₉ is hydrogen, alkyl containing 1-3 carbon atoms or halo. It is even more preferred that R₉ is hydrogen.

When the compound utilized is an androstane, both R₉ and R₁₀ are present on the ring. It is preferred that R₉ and R₁₀ are independently hydrogen, alkyl containing 1-3 carbon atoms, especially methyl, alkoxy containing 1-3 carbon atoms, especially methyl, halo, especially fluoro or chloro or hydroxy. It is most preferred that one of R₉ and R₁₀ is hydrogen and the other is as defined hereinabove.

In the preferred embodiment, the carbon atoms to which R₁, R₂, R₃, R₄, R₇, R₈, R₉, R₁₀, R₁₁, R₁₂, R₁₃, R₁₄ or R₁₅ are either unsubstituted or monosubstituted. In other words, in the preferred embodiment, at least one of R₁ and R₂ is hydrogen, and at least one of R₃ and R₄ is hydrogen, and at least one of R₇ and R₈ is hydrogen, and at least one of R₁₁ and R₁₂ is hydrogen and at least one of R₁₃ and R₁₄ is hydrogen. When the compound utilized is in androstene, R₉ can have any of the values indicated hereinabove; however, when the compound utilized is an androstene, in the preferred embodiment, at least one of R₉ and R₁₀ is hydrogen.

In the most preferred embodiment, either all of R₁, R₂, R₃, R₄, R₇, R₈, R₉, R₁₀ (when present), R₁₁, R₁₂, R₁₃, R₁₄ or R₁₅ are all hydrogen, or one of R₁, R₂, R₃, R₄, R₇, R₈, R₉, R₁₀ (when present), R₁₁, R₁₂, R₁₃, R₁₄, is a non-hydrogen substituent and the rest are hydrogen. In the latter case, it is preferred that only one of R₉, R₁₀, R₁₁ or R₁₂ is substituted, as defined herein, and R₁, R₂, R₃, R₄, R₇, R₈, R₁₃, R₁₄ and R₁₅ are hydrogen.

It is preferred that R₅ and R₆ in both Formula I and II are other than hydroxy. It is preferred that R₅ and R₆ are independently hydrogen, lower alkyl, especially alkyl containing 1-3 carbon atoms, or halo, especially chloro or fluoro. In the most preferred embodiment, R₅ and R₆ are independently lower alkyl, especially alkyl containing 1-3 carbon atoms or hydrogen. It is even more preferred that R₅ and R₆ are independently hydrogen or methyl. In the most preferred embodiment, R₆ is hydrogen and R₅ is hydrogen, alkyl containing 1-3 carbon atoms, especially methyl or halo, especially fluoro or chloro. It is even more preferred that R₆ is hydrogen and R₅ is hydrogen or alkyl containing 1-3 carbon atoms, especially methyl. It is most preferred that both R₅ and R₆ are hydrogen.

In the definitions hereinabove, R₁₆ and R₁₇ can have any of the aforementioned values. However, the present inventor has found that when R₁₆ is either an alkenyl or alkynyl, R₁₇ cannot also be an alkenyl or alkynyl and vice versa. In other words. only one of R₁₆ and R₁₇ can contain an alkenyl or alkynyl group, if present.

It is preferred that R₁₆ and R₁₇ are independently hydrogen, lower alkyl, lower alkoxy, or hydroxy or halo, especially chloro and most especially fluoro. In a more preferred embodiment, R₁₆ and R₁₇ are independently hydrogen, alkyl containing 1-3 carbon atoms, alkoxy containing 1-3 carbon atoms, hydroxy or halo, especially chloro and most especially fluoro. It is even more preferred that R₁₆ and R₁₇ are independently hydrogen or halo, especially chloro or fluoro.

It is especially preferred that at least one of R₁₆ and R₁₇ is other than hydrogen. It is most preferred that R₁₇ is hydrogen and R₁₆ is other than hydrogen. In the more especially preferred embodiment, R₁₇ is hydrogen and R₁₆ is halo, especially chloro and most especially fluoro, lower alkyl, especially alkyl containing 1-3 carbon atoms, hydroxy, lower alkoxy, especially alkoxy containing 1-3 carbon atoms, or hydroxy. In the even more preferred embodiment, R₁₇ is hydrogen and R₁₆ is halo, especially chloro and most especially fluoro, methyl, methoxy or hydroxy.

It is most especially preferred that R₁₇ is hydrogen and R₁₆ is halo, especially chloro or fluoro. It is most especially preferred that R₁₇ is hydrogen and R₁₆ is fluoro.

Moreover, in the compounds of Formula I and II it is preferred that the hydrogen atom in the 8 position is β. it is also preferred that the hydrogen atom in the 14 position is alpha. The substituent on the C-9(R₁₅) may be α or β configuration. It is indicated herein by a wavy line. It is preferred that the R₁₅ substituent is alpha.

Preferred compounds of Formula I have the formula wherein R₅ and R₆ are both hydrogen and the other variables are as defined above. In this embodiment, it is preferred that R₁, R₂, R₃, R₄, R₅, R₆, R₇, R₈, R₁₁, R₁₂, R₁₃, R₁₄ and R₁₅ are independently hydrogen, alkyl, halogen, or alkoxy or hydroxy, R₉ is hydrogen alkyl or halogen and R₁₆ is lower alkyl, halo, hydroxy or lower alkoxy and R₁₇ is hydrogen, lower alkyl, halo, hydroxy or lower alkoxy. It is most preferred that R₅ and R₆ are hydrogen, that R₁₇ is hydrogen, R₁, R₂, R₃, R₄, R₇, R₈, R₁₃, R₁₄ and R₁₅ are hydrogen lower alkyl or halo and R₉ is hydrogen or lower alkyl and R₁₁ and R₁₂ are independently hydrogen, halo, lower alkyl or hydroxy. It is even more preferred that R₅ and R₆ are hydrogen and R₁-R₄ and R₇-R₁₅ are all hydrogen and R₁₆ is halo, especially fluoro and R₁₇ is hydrogen.

In another embodiment, it is preferred that R₅ and R₆ are hydrogen and R₁₆ is fluoro and R₁₇ is hydrogen. R₁-R₄ are all hydrogen and R₁, R₂, R₃, R₆, R₇, R₈, R₉, R₁₁, R₁₂, R₁₃, R₁₄ and R₁₅ are independently hydrogen, alkyl, halogen or alkoxy or hydroxy, R₉ is hydrogen and alkyl or halogen. In this embodiment wherein R₅ and R₆ are hydrogen, R₁₆ is fluoro and R₁₇ is hydrogen it is even more preferred that R₁, R₂, R₃, R₄, R₇, R₈, R₁₃, R₁₄ and R₁₅ are hydrogen or lower alkyl, R₁₁ is hydrogen and R₁₂ is hydrogen. In the most preferred embodiment, R₁-R₁₅ and R₁₇ are hydrogen and R₁₆ is fluoro.

Preferred compounds of Formula II have the formula wherein R₅ and R₆ are both hydrogen and the other variables are as defined hereinabove. In this embodiment, it is preferred that R₁, R₂, R₃, R₄, R₅, R₆, R₇, R₈, R₁₁, R₁₂, R₁₃ and R₁₄ and R₁₅ are independently hydrogen, alkyl, halogen, hydroxy or alkoxy, R₉ and R₁₀ are independently hydrogen, alkyl or halogen; and R₁₆ is lower alkyl, halo, hydroxy, or lower alkoxy and R₁₇ is hydrogen, lower alkyl, halo, hydroxy or lower alkoxy. It is more preferred that R₅ and R₆ are hydrogen, R₁₇ is hydrogen, R₁, R₂, R₃, R₄, R₇, R₈, R₁₃, R₁₄ and R₁₅ are independently hydrogen, lower alkyl, halo or hydroxy, R₉ and R₁₀ are independently hydrogen or lower alkyl and R₁₁ and R₁₂ are independently hydrogen, halo, lower alkyl or hydroxy. It is even more preferred that R₁-R₁₅ and R₁₇ are hydrogen and R₁₆ is halo, especially fluoro.

In another embodiment, it is preferred that R₅ and R₆ are hydrogen. R₁₆ is fluoro, R₁₇ is hydrogen and R₁, R₂, R₃, R₄, R₇, R₈, R₁₃, R₁₄ and R₁₅ are independently hydrogen, alkyl, halogen or alkoxy or hydroxy and R₉ and R₁₀ are independently hydrogen, alkyl, halogen or hydroxy. In this embodiment wherein R₅ and R₆ are hydrogen, R₁₆ is fluoro and R₁₇ is hydrogen, it is even more preferred that R₁, R₂, R₃, R₄, R₇, R₈, R₁₃, R₁₄ and R₁₅ are hydrogen or lower alkyl, and R₁₁ and R₁₂ are hydrogen. In the most preferred embodiment, R₁-R₁₅ and R₁₇ are hydrogen and R₁₆ is fluoro.

Preferred compounds of the Formula I have the formula:

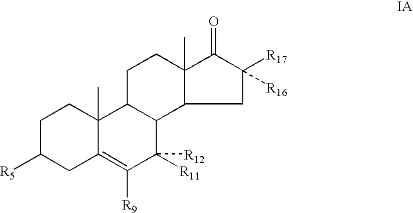

wherein

R₅ is hydrogen or lower alkyl;

R₉ is hydrogen or halo or lower alkyl;

R₁₁ and R₁₂ are independently hydrogen, lower alkyl, hydroxy, lower alkoxy, or halo;

R₁₇ is hydrogen, lower alkyl or halo, especially fluoro; and

R₁₆ is hydroxy, lower alkyl, lower alkoxy or halo, especially chloro and most especially fluoro.

In this embodiment, it is preferred that R₅ is hydrogen or methyl and especially hydrogen. It is also preferred that R₉ is hydrogen. The preferred embodiments of R₁₁ and R₁₂ are hydrogen, methyl, hydroxy or methoxy or halo, the most preferred halo being chloro and most especially fluoro.

It is preferred that R₁₆ is halo, especially chloro and most especially fluoro.

The preferred values of R₁₇ is hydrogen, methyl or halo, the most preferred halo being fluoro. It is more preferred that R₁₇ is fluoro and most especially hydrogen.

Preferred compounds of Formula II have the formula:

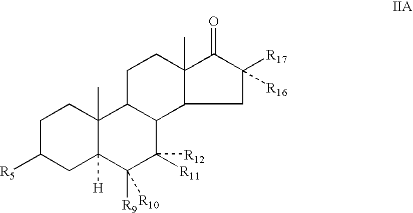

wherein

R₅ is hydrogen or lower alkyl;

R₉ is hydrogen or halo or lower alkyl;

R₁₁ and R₁₂ are independently hydrogen, lower alkyl, hydroxy, lower alkoxy, or halo; and

R₁₇ is hydrogen, lower alkyl or halo, the most preferred halo being fluoro; and

R₁₆ is hydroxy, lower alkyl, lower alkoxy or halo, especially chloro and most especially fluoro.

In this embodiment, it is preferred that R₃ is hydrogen or methyl and especially hydrogen.

It is preferred that R₉ and R₁₀ are independently hydrogen, methoxy, methyl or halogen, especially chloro and most especially fluoro. It is most preferred, however, that R₉ and R₁₀ are hydrogen.

The preferred embodiments of R₁₁ and R₁₂ are hydrogen, methyl, hydroxy, methoxy or halo, especially chloro and most especially fluoro.

It is preferred that R₁₆ is halo, especially chloro and most especially fluoro.

The preferred values of R₁₇ is hydrogen, methyl or halo, especially fluoro. It is especially preferred that R₁₇ is fluoro and most especially hydrogen.

It is to be understood that in the formulae depicted hereinabove, the various combinations and permutations of the various definitions of R₁-R₁₇ are contemplated to be within the scope of the compounds utilized in the present invention.

Preferred compounds for use in the present invention include:

1α-methyl-5-androsten-17-one,

2α,6,16α-trimethyl-5-androsten-17-one,

16α-ethynyl-6-chloro-5-androsten-17-one,

3β-methyl-5-androsten-17-one,

3β-ethyl-5-androsten-17-one,

3β-butyl-5-androsten-17-one,

6,16α-dimethyl-5-androsten-17-one,

2α,7β-dimethyl-5-androsten-17-one,

1α-chloro-3β-methyl-5-androsten-17-one,

4α-methyl-5-androsten-17-one,

3β-methyl-7β-chloro-5-androsten-17-one,

3β-methyl-16α-ethyl-5-androsten-17-one,

3β-methyl-16α-ethynyl-5-androsten-17-one,

3β,16α,16β-trimethyl-5-androsten-17-one,

3β-methyl, 16α,16β-difluoro-5-androsten-17-one,

2α,3β-dimethyl-5-androsten-17-one,

3β,4α,7β-trimethyl-5-androsten-17-one,

2α,3β,6-trimethyl-5-androsten-17-one,

3β,4α,7β-trimethyl-5-androsten-17-one,

6-methyl-5-androsten-17-one,

7β-methyl-5-androsten-17-one,

16α-fluoro-3β-methyl-5-androsten-17-one,

16α-methoxy-5-androsten-17-one,

11α-methyl-5-androsten-17-one,

16α-methyl-5-androsten-17-one,

3β,16β-dimethyl-5-androsten-17-one,

16α-hydroxy-3β-methyl-5-androsten-17-one,

16β-fluoro-5-androsten-17-one,

3β-hydroxy-16α-fluoro-5-androsten-17-one,

16α-fluoro-3β-methyl-5-androsten-17-one,

16α-fluoro-3β,16β-dimethyl-5-androsten-17-one,

16α-fluoro-16β-methyl-5-androsten-17-one,

3β-methyl-5α-androstan-17-one,

3β-methyl-7α-chloro-5α-androstan-17-one,

3β-methyl-16α-ethyl-5α-androstan-17-one,

3β-methyl-16α-ethynyl-5α-androstan-17-one,

2α,3β-dimethyl-5α-androstan-17-one,

3β,4α-dimethyl-5α-androstan-17-one,

1α-methyl-5α-androstan-17-one,

3β,16α-dimethyl-5α-androstan-17-one,

16α-hydroxy-3β-methyl-5α-androstan-17-one,

16α-fluoro-3β-methyl-5α-androstan-17-one,

16α-hydroxy-3β-methyl-5α-androstan-17-one,

16α-fluoro-3β-methyl-5α-androstan-17-one,

16α-fluoro-3β,16β-dimethyl-5α-androstan-17-one,

16α-fluoro-3β,16β-dimethyl-5α-androstan-17-one,

3β,16α,16β-trimethyl-5α-androstan-17-one,

3β-methyl-16α,16β-difluoro-5α-androstan-17-one,

16α-hydroxy-5-androsten-17-one,

16α-fluoro-5-androsten-17-one,

16α-fluoro-16β-methyl-5-androsten-17-one,

16α-methyl-5-androsten-17-one,

16β-methyl-5-androsten-17-one,

16α-hydroxy-5α-androstan-17-one,

16α-fluoro-5α-androstan-17-one,

3β-hydroxy-16α-fluoro-5α-androstane-17-one

16α-fluoro-16β-methyl-5α-androstan-17-one,

16α-methyl-5α-androstan-17-one,

16α-fluoro-7α-hydroxy-5-androsten-17-one,

16α-fluoro-7α-hydroxy-5α-androstan-17-one,

16α-fluoro-7β-hydroxy-5-androsten-17-one,

16α-fluoro-7β-hydroxy-5α-androstan-17-one,

16α-methoxy-5α-androstan-17-one,

3β-methyl-16α-fluoro-7-hydroxy-5-androsten-17-one, and

3β-methyl-16α-fluoro-7-hydroxy-5α-androstan-17-one.

The compounds of the present invention can be prepared by art-recognized techniques from known compounds or readily preparable intermediates. Exemplary procedures are described in U.S. Pat. Nos. 5,804,576, 5,744,462, 5,714,481, 5,700,793, 5,696,106, 5,656,621, 5,157,031 and 5,001,119, the contents of all of which are incorporated by reference. If substituents on the steroidal ring are themselves reactive under the reaction conditions, then these substituents can themselves be protected utilizing protecting groups according to chemical techniques known in the art. A variety of protecting groups known in the art may be employed. Examples of many of these possible groups can be found in “Protective Groups in Organic Synthesis”, by J. W. Green, John Wiley and Sons, 1981.

If more than one substituent is to be added to the steroidal ring, the substituents can be added in any order except that it is preferred that the halogens are added last.

Finally, it should be noted that the procedures described in the aforementioned patents are applicable to all of the steroids contemplated to be utilized in the present invention, regardless of whether a double bond is present at the 5,6 position of the steroidal ring.

Moreover, the steroids of Formula II can be prepared from the corresponding steroids of Formula I by techniques known to one skilled in the art, e.g., by catalytic hydrogenation using, e.g., H₂/Pd, H₂/Pt or H₂/Ni.

The compounds utilized in the present method are used in therapeutically effective amounts.

The physician will determine the dosage of the present therapeutic agents which will be most suitable and it will vary with the form of administration and the particular compound chosen, and furthermore, it will vary depending upon various factors, including but not limited to the patient under treatment and the age of the patient, the seventy of the condition being treated and the like. He will generally wish to initiate treatment with small dosages substantially less than the optimum dose of the compound and increase the dosage by small increments until the optimum effect under the circumstances is reached. It will generally be found that when the composition is administered orally, larger quantities of the active agent will be required to produce the same effect as a smaller quantity given parenterally. The compounds are useful in the same manner as comparable therapeutic agents and the dosage level is of the same order of magnitude as is generally employed with these other therapeutic agents. When given parenterally, the compounds are administered generally in dosages of, for example, about 0.1 to about 10 mg/kg/day, also depending upon the host and the severity of the condition being treated and the compound utilized.

In a preferred embodiment, the compounds utilized are orally administered in amounts ranging from about 4 mg to about 35 mg per kilogram of body weight per day, depending upon the particular mammalian host and more preferably from about 6 to about 28 mg/kg body weight per day. This dosage regimen may be adjusted by the physician to provide the optimum therapeutic response. For example, several divided doses may be administered daily or the dose may be proportionally reduced as indicated by the exigencies of the therapeutic situation.

The compounds of Formulae I or II may be administered in a convenient manner, such as by oral, intravenous, intramuscular or subcutaneous routes.

The compounds of Formula I or II may be orally administered, for example, with an inert diluent or with an assimilable edible carrier, or it may be enclosed in hard or soft shell gelatin capsules, or it may be compressed into tablets, or it may be incorporated directly into the food of the diet. For oral therapeutic administration, the compounds of Formula I or II may be incorporated with excipients and used in the form of ingestible tablets, buccal tablets, troches, capsules, elixirs, suspensions, syrups, wafers, and the like. Such compositions and preparations should contain at least 1% of active compound of Formula I or II. The percentage of the compositions and preparations may, of course, be varied and may conveniently be between about 5 to about 80% of the weight of the unit. The amount of the compound of Formula I or II used in such therapeutic compositions is such that a suitable dosage will be obtained. Preferred compositions or preparations according to the present invention contain between about 200 mg and about 4000 mg of active compound of Formula I or II.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the active compound, sucrose as a sweetening agent, methyl and propyl parabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations. For example, sustained release dosage forms are contemplated wherein the active ingredient is bound to an ion exchange resin which, optionally, can be coated with a diffusion barrier coating to modify the release properties of the resin or wherein the active ingredient, i.e., a compound of Formula I or II, is associated with a sustained release polymer known in the art, such as hydroxynronylmethylcellulose and the like

The active compound may also be administered parenterally. It is especially advantageous to formulate parenteral compositions in dosage unit form for ease of administration and uniformity of dosage. Dispersions can also be prepared in glycerol, liquid polyethylene glycols, e.g., PEG 100, PEG 200, PEG 300, PEG 400, and the like, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form is usually sterile and must be fluid to the extent that syringability exists. It must be stable under the conditions of manufacture and storage and usually must be preserved against the contaminating action of microorganisms such as bacteria and fungi. The carrier can be a solvent or dispersion medium containing, for example, water, ethanol, polyol (for example, glycerol, propylene glycol, and one or more liquid polyethylene glycol, e.g. as disclosed herein and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders, the above solutions are vacuum dried or freeze-dried, as necessary.

The compounds of Formula I or Formula II can also be applied topically, as e.g., through a patch using techniques known to one of ordinary skill in the art.

The active ingredients, that is a compound of Formula I and/or II, can be administered buccally by preparing a suitable formulation of the compounds of the present invention and utilizing procedures well known to those skilled in the art. These formulations are prepared with suitable non-toxic pharmaceutically acceptable ingredients. These ingredients are known to those skilled in the preparation of buccal dosage forms. Some of these ingredients can be found in Remington's Pharmaceutical Sciences, 17^{th} edition, 1985, a standard reference in the field. The choice of suitable carriers is highly dependent upon the exact nature of the buccal dosage form desired, e.g., tablets, lozenges, gels, patches and the like. All of these buccal dosage forms are contemplated to be within the scope of the present invention and they are formulated in a conventional manner. Preferably, an effective amount of active ingredient in the buccal form ranges from about 0.15 mg/Kg to 3.5 mg/Kg. For example, the buccal dosage form comprises the compound of Formula I or II, e.g., 16α-fluoro-5-androsten-17-one or 16α-fluoro-5α-androstan-17-one in therapeutically effective amounts, as defined herein in association with a pharmaceutically acceptable polymer carrier.

Preferably, the pharmaceutically acceptable polymer carrier is a polymer that adheres to the wet surface of the buccal mucosa and is bioerodible. It is described in more detail hereinbelow. In one embodiment, the buccal dosage form comprises the compounds of Formula I or II in effective amounts and the polymer. However, other excipients may optionally be present, e.g., binders, disintergrants, lubricants, diluents, flavorings, colorings, and the like.

Ideally, the carrier comprises a polymer having sufficient tack to ensure that the dosage unit adheres to the buccal mucosa for the necessary time period, i.e., the time period during which the compounds of Formula I and II are to be delivered to the buccal mucosa. Additionally, it is preferred that the polymeric carrier is gradually bioerodible, i.e., the polymer hydrolyzes at a predetermined rate upon contact with moisture. The polymeric carrier is preferably sticky when moist, but not when dry, for convenience in handling. Generally, it is preferred that the average molecular weight of the polymer range from about 4,000 to about 1,000,000 g. One of skill in the art will appreciate that the higher the molecular weight of the polymer the slower the erosion time.

Any polymeric carrier can be used that are pharmaceutically acceptable, provide both a suitable degree of adhesion and the desired drug release profile and are compatible with the agents to be administered and any other components that may be present in the buccal dosage unit. Generally, the polymeric carriers comprise hydrophilic (water-soluble and water-swellable) polymers that adhere to the wet surface of buccal mucosa. Examples of polymeric carrier useful herein include acrylic acid polymers and copolymers, e.g., those known as “carbomers” (Carbopol™ which may be obtained from GAF); vinyl polymers and copolymers; polyvinyl pyrrolidone, dextran, guar gum, pectins, starches; and cellulose polymers, such as hydroxypropyl methylcellulose (e.g., Methocel® obtainable from Dow Chemical Company), hydroxypropyl cellulose (e.g., Klucel™, which may also be obtained from Dow), hydroxypropyl cellulose ethers (see, e.g., U.S. Pat. No. 4,704,285 to Alderman), hydroxyethyl cellulose, sodium carboxymethyl cellulose, methyl cellulose, ethyl cellulose, cellulose acetate phthalate, cellulose acetate butyrate, and the like. The carrier may also comprise two or more suitable polymers in combination, for example, a carbomer combined in approximately 1:5 to 5:1 ratio, by weight, with a polyethylene oxide.

The present dosage unit comprises the active agent and the polymeric carrier. However, it may be desirable in some cases to include one or more additional components. For example, a lubricant may be included to facilitate the process of manufacturing the dosage units; lubricants may also optimize erosion rate and drug flux. If a lubricant is present, it will represent on the order of 0.01 wt. % to about 2 wt. %, preferably about 0.01 wt. % to 0.5 wt, %, of the dosage unit. Suitable lubricants include, but are not limited to, magnesium stearate, calcium stearate. stearic acid, sodium stearylfulmarate, talc, hydrogenated vegetable oils and polyethylene glycol. As will be appreciated by those skilled in the art, however, modulating the particle size of the components in the dosage unit and/or the density of the unit can provide a similar effect—i.e., improved manufacturability and optimization of erosion rate and drug flux—without addition of a lubricant.

Other components may also optionally be incorporated into the buccal dosage unit. Such additional optional components include, for example, one or more disintegrants, diluents, binders, enhancers, or the like. Examples of disintegrants that may be used include, but are not limited to, cross linked polyvinylpyrrolidones, such as crospovidone (e.g., Polyplasdone® XL, which may be obtained from GAF), cross-linked carboxylic methylcelluloses, such as croscanmelose (e.g., Ac-di-sol®, which may be obtained from FMC), alginic acid, and sodium carboxymethyl starches (e.g., Explotab®, which may be obtained from Edward Medell Co., Inc.), agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by crystallization with dextrin (e.g., co-crystallized sucrose and dextrin such as Di-Pak®, which may be obtained from Amstar), lactone, calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Permeation enhancers may also be present in the novel dosage units in order to increase the rate at which the active agents pass through the buccal mucosa. Examples of permeation enhancers include, but are not limited to, dimethylsulfoxide (“DMSO”), dimethylformamide (“DMF”), N,N-dimethylacetamide (“DMA”), decylmethylsulfoxide (“C₁₀MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone® from Nelson Research & Development Co., Irvine, Calif.), lower alkanols (e.g., ethanol), SEPA® (available from Macrochem Co., Lexington, Mass.) cholic acid, taurocholic acid, bile salt type enhancers, and surfactants such as Tergitol®, Nonoxynol-9® and TWEEN-80®.

Flavorings may be optionally included in the buccal formation. Any suitable flavoring may be used, e.g., mannitol, lactose or artificial sweeteners such as aspartame. Coloring agents may be added, although again, such agents are not required. Examples of coloring agents include any of the water-soluble FD&C dyes, mixtures of the same, or their corresponding lakes.

In addition, if desired, the present dosage units may be formulated with one or more preservatives or bacteriostatic agents, e.g., methyl hydroxybenzoate, propyl hydroxybenzoate, chlorocresol, benzalkonium chloride, or the like.

In general, the dosage unit of the invention is compositionally a substantially homogeneous, substantially uniform formulation. By “substantially uniform” is meant that the dosage unit is not coated, does not contain a plurality of layers or other types of discrete segments. Rather, the substance of the dosage unit is similar throughout, so that the unit is essentially “monolithic” in nature.

The buccal dosage units may be in the form of tablets made by either conventional compression or molding methods. See, e.g., Remington's Pharmaceutical Sciences, 18th edition (Easton, Pa.: Mack Publishing Co., 1990). Preferably, the dosage units are prepared by mixing the components together and compressing the mixture into tablet form. As will be appreciated by those skilled in the art, the erosion rate of the dosage unit, and thus the rate of drug delivery, is controlled by three factors: the pressure used to make the tablets, and thus the tablets' density; the carrier selected, as alluded to above; and the carrier-to-drug ratio. Pressure, carrier and carrier-to-drug ratio may thus be varied to obtain shorter acting or longer-lived dosage units.

The dosage units may have any of the conventional shapes, for example, lozenges, disks, wafers, tablets or the like.

The buccal dosage units may also be generated by a molding process. Preferably, the final unit should have a melting point which is high enough to prevent fusion of packaged dosage units during shipping and storage, yet low enough to permit mixing of pharmaceutical ingredients without significant decomposition of the active agents when being incorporated into the molten carrier.

The most preferred mode of administration is the buccal form. The preferred buccal form is a tablet, and more preferably, a tablet containing fluasterone. In a preferred embodiment, the buccal tablet comprises by weight 16% fluasterone, 72% mannitol, 7% crospovidone, 2% magnesium stearate, 1% polyethylene glycol, e.g. PEG 3350, 1% sodium lauryl sulfate and 1% amorphous silica dioxide. Without wishing to be bound, it is believed that the buccal form of administration avoids the disadvantages encountered with oral drug administration, e.g., degradation of the steroid by fluids present in the gastrointestinal tract and/or first-pass inactivation in the liver and/or intestines. Moreover, unlike the oral form, the administration of the drug buccally enhances the efficacy thereof relative to oral administration. Further, it decreases the androgenicity of the drug, as compared to the oral mode of administration. This is important, especially since increased androgenicity counteracts the anti-diabetic effect of the drug. In addition, oral administration of steroids tends to lower HDL (high density lipoproteins) in men and women, an effect that is most undesirable. However, when the compounds of Formula I and II are administered buccally, these androgenic side effects, such as HDL lowering observed when the drug is given in high concentration during oral therapy, is significantly reduced, if not eliminated.

As used herein, “pharmaceutically acceptable carrier” includes any and all solvents, dispersion media, coatings, antibacterial and antifuingal agents, isotonic and absorption delaying agents for pharmaceutical active substances well known in the art. Except insofar as any conventional media or agent is incompatible with the active ingredient, their use in the therapeutic compositions is contemplated. Supplementary active ingredients can also be incorporated into the compositions.

Dosage unit form as used herein refers to physically discrete units suited as unitary dosages for the subjects to be treated; each unit containing a predetermined quantity of active material calculated to produce the desired therapeutic effect in association with the required pharmaceutical carrier.

The principal active ingredient is compounded for convenient and effective administration in effective amounts with a suitable pharmaceutically acceptable carrier in dosage unit form as hereinbefore described. A unit dosage, for example, contains the principal active compound in amounts ranging from about 10 mg e.g. in humans, or as low as 1 mg (for small animals) to about 2000 mg. If placed in solution, the concentration of the compounds of Formula I or Formula II preferably ranges from about 10 mg/mL to about 250 mg/mL. In the case of compositions containing supplementary active ingredients, the dosages are determined by reference to the usual dose and manner of administration of the said ingredients. In the case of buccal administration, the compounds of Formula I or II are preferably in the buccal unit dosage form present in an amount ranging from about 10 to about 50 mg. As used herein the term “patient” or “subject” refers to a warm blooded animal, and preferably mammals, such as, for example, cats, dogs, horses, cows, pigs, mice, rats and primates, including humans. The preferred patient is humans.

The compounds described hereinabove are useful in the treatment of patients having hypertriglyceridemia. It is most effective when the triglyceride concentration in the plasma is greater than about 200 mg/dl, as described hereinbelow.

The term “treat” when referring to patients having hypertriglyceridemia refers to reducing the plasma triglycerides of the patient in a detectable amount. Such reduction may be, e.g. a reduction of about 10%, 20%, 30% or 40% in a patient's plasma triglyceride level. When referring to other diseases, it refers to the management and care of a mammalian subject, preferably human, for the purpose of combating the disease, condition or disorder, and includes the administration of a compound of the present invention to complications or eliminating the disease, condition or disorder.

The preferred patient population to be treated by the method of the present invention includes diabetic patients having type II diabetes mellitus and hyperlipidemia and/or hyperlipidemics who are non-diabetic but have insulin-resistance.

The compounds utilized herein lower the triglyceride concentration in the patient, suffering from hypertriglyceridemia. The compounds described herein are effective in treating patients having a concentration of free plasma triglycerides of greater than about 200 mg/dl and preferably greater than or equal to about 300 mg/dl and especially greater than or equal to about 500 mg/dl.

They are especially effective in treating patients having hypertriglyceridemia, who also have a low HDL level in the plasma. As used herein, low “HDL levels” refer to the concentration of HDL cholesterol in the plasma of less than about 40 mg/dl for men and less than about 45 mg/dl for women. It is even more effective in treating men having an HDL level less than 35 mg/dl and women having HDL levels less than about 40 mg/dl.

The compounds of Formula I and II herein are especially effective in reducing the triglyceride concentration in patients having hypertriglyceridemia who are also obese patients. The term “obese” and “obesity” refers to a patient, e.g., humans, having a body mass index (BMI) greater than 30 kg/m² BMI, by definition, equals weight (kg)/height² (m²). Often times, an obese patient also has low levels of HDL, as defined herein and/or is insulin resistant.

In addition, the compounds of Formula I and II herein are also effective in reducing the triglyceride concentration in patients who are insulin resistant.

The term “insulin resistance” can be defined generally as a disorder generally a disorder of glucose metabolism. More specifically, insulin resistance can be defined as the diminished ability of insulin to exert its biological action across a broad range of concentrations producing less than expected biologic effect. (see, e.g., Reaven, G. M., J. Basic & Clin. Phys. & Pham. (1998) 9: 387-406 and Flier, J. Ann Rev. Med. (1983) 34:145-60). Insulin resistant persons have a diminished ability to properly metabolize glucose and require insulin therapy. Manifestations of insulin resistance include insufficient insulin activation of glucose uptake, oxidation and storage in muscle and inadequate insulin repression of lipolysis in adipose tissue and of glucose production and secretion in liver. Insulin resistance can cause or contribute to polycystic ovarian syndrome, Impaired Glucose Tolerance (IGT), gestational diabetes, hypertension, obesity, atherosclerosis and a variety of other disorders. Eventually, the insulin resistant individuals can progress to a point where a diabetic state is reached. The association of insulin resistance with glucose intolerance, an increase in plasma triglyceride and a decrease in high-density lipoprotein cholesterol concentrations, high blood pressure, hyperuricemia, smaller denser low-density lipoprotein particles, and higher circulating levels of plaminogen activator inhibitor-1), has been referred to as “Syndrome X” (see, e.g., Reaven, G. M., Physiol. Rev. (1995) 75: 473-486).

The compounds of Formula I and II are useful in modulating insulin resistance in a patient, e.g., mammal, the method comprising administering to the patient a therapeutically effective amount of the compound of Formula I or Formula II. Insulin resistance, may be a marker for generalized lipodystrophies. Thus, the compounds of the present invention are useful in treating lipodystrophies. Lypodystrophies have been known for over, a century and are characterized by selective loss of body fat that can vary from small indentation or depressed areas in patients with localized lipodystrophies to near complete absence of adipose tissue in generalized lipodystrophies. More particularly, the compounds of Formula I and II are useful in treating generalized lipodystrophies.

The amounts of compounds of Formula I and II used in the treatment are therapeutically effective amounts, as described hereinabove. In treating this malady, the compounds of Formula I and TI can be administered to the patient using any of the modes of

Thus, the steroids of the Formula I and II are useful for treating and lowering the triglyceride levels in patients having hypertriglyceridemia and more preferably having, in addition at least one of the following characteristics:

(a) obese, especially those having a BMI>30;

(b) low HDL; and

(c) insulin-resistant.

In addition, it is preferred that the patient is less than forty years of age.

By administering a therapeutically effective amount of the compounds described hereinabove to such patients, the compounds of the present invention reduces the plasma triglycerides, as shown hereinbelow.

The compounds of Formula I and II described herein are each effective in treating hypertriglyceridemic patients.

In addition the compounds of Formula I and II are each effective in treating hypertriglyceridemic patients having at least one of the following traits: low HDL, or insulin resistance or patients which are obese, those having a BMI greater than 30. It is preferred that the patient is less than forty years of age. The compounds used in the present invention are useful for treating hypertriglyceridemics which exhibit none or one, two or three of these traits.

Moreover, the compounds of Formula I and II are each useful for the treatment of Syndrome-X, also known as the insulin resistance syndrome. It includes hyperlipidemia, hyperinsulinemia, obesity, insulin resistance, insulin resistance leading to type-2 diabetes and diabetic complications thereof, i.e., diseases in which insulin resistance is the pathophysiological mechanism.

Moreover, the compounds of Formula I and II are useful for treating hypertriglyceridemia, hypertension and coronary artery disease.

The compounds of Formula I and II are also useful in treating familial combined hyperlipidemia. Familial combined hyperlipidemia is a common disorder in which affected individuals have either hypercholesterolemia, hypercholesterolemia with hypertriglyceridemia or hypertriglyceridemia. These individuals are prone to premature atherosclerosis and coronary heart disease.

Patients with hypertriglyceridemia insulin resistance, low HDL and/or obesity having BMI's greater than 30 are prone to suffering from atherosclerosis and coronary heart disease and/or stroke. Thus, the present invention is directed to a method of treating or preventing atherosclerosis or stroke resulting from hypertriglyceridemia by administering to said mammalian species in need of treatment a therapeutically effective amount of compound I or II.

As used herein and unless stated otherwise, the term prophylaxis”, “prevent” and the like means reducing the risk of a patient which is prone or at a risk of contracting a disease or medical condition described in the instant application. Moreover, the term “at risk of contracting” refers to a patient who has been identified as being exposed to or as having one or more risk factors associated with the onset of disease. These risk factors may be environmental, genetic or biological.

The term “ameliorate” one or more symptoms or the like means to reduce the severity or improve or to mask one or more symptoms of the disease.

The compounds of the present invention have beneficial effects on the risk factors for the development of cardiovascular disease, type-2-diabetes, vascular disease and stroke. It is believed, without wishing to be bound, that elevated levels in the plasma of acute phase proteins and inflammatory cytokines, such as C-ractive proteins, interleukin-6, Pa AI-1, or TNFα and the like are sensitive markers for systemic inflammation and for the development of cardiovascular disease, type-2-diabetes, vascular disease and stroke. Without wishing to be bound, it is believed that elevated amounts (relative to normal) are markers and/or present during the development of these diseases. By elevated amounts, it is meant that their concentrations in the plasma are greater than normal levels. For example, elevated levels of C-reactive proteins are present in the plasma in concentrations greater than 1.15 mg/l for both men and women. These amounts can be measured and determined using standard techniques known to one skilled in the art. Without wishing to be bound, it is believed that compounds of Formula I and II depress plasma levels of these, one or more of cytokines and acute phase proteins, such as C-reactive proteins, and the like, especially if given in effective doses, as defined herein. Thus, the treatment utilizing compounds of Formula I and II reduce the development or severity of cardiovascular disease and stroke.

Another preferred embodiment of the present invention is to use the compounds of Formula I or II to lower the abnormal levels of C-reactive protein IL-6, Pa AI-1, or TNFα in patients having high levels of C-reactive proteins.

Without wishing to be bound, it is believed that the compounds of Formula I and II of the present invention behave by two possible mechanisms.

It has been found that hypercortisolism, an exceedingly high concentration of hydrocortisone, a glucocorticoid found in humans, directly contributes to the phenotype and metabolic abnormalities of the metabolic syndrome (Syndrome X), including obesity, insulin resistance, and hypertiglyceridemia. (See, Peeke, et al., Annals. NY Acad. Sci., 771, 665-676 (1995). The steroids of Formula I and II have an antiglucorticoid effect. Thus, it is believed, without wishing to be bound, that the compounds of the present invention reduce the hypercortisolism.

Without wishing to be bound, it is also believed that a state of chronic subclinical inflammation also directly contributes to the phenotypic and metabolic abnormalities of Syndrome X. In addition, it is believed that proinflammatory cytokines may act directly to induce insulin resistance and hypertriglyceridemia or act indirectly through the stimulation of cortisol production. The steroids of Formula I and II are anti-inflammatory agents, and this contributes to the selective triglyceride lowering effect in obese patients.

The compounds of Formula I and II are also used to reduce the enhance glucocorticoid activity or actions in an animal, e.g. mammal.

Enhanced glucocorticoid action has been implicated as a cause for or as being associated with a number of ailments affecting animals, including mammals, especially man. For example, individuals may be immunosuppressed as a consequence of endogenous elevations in adrenal glucocorticoid (GCS) levels. These elevated levels can result from a variety of causes, including, but not limited to, stress and trauma (including, for example, post surgical trauma, and burn trauma), as a secondary consequence to any clinical condition which causes an elevated production of interleukin-1 (IL-1) or therapeutic treatment for a variety of clinical conditions. These elevated GCS levels can result in an imbalance in the production of essential interleukins. As a consequence thereof, the animals exhibit a depressed capacity to produce species of lymphokines which are essential to the development of protective forms of immunity. Plasma glucocorticoid steroid levels can also be elevated exogenously as a consequence of therapeutic treatment for a variety of clinical conditions. In addition to the above, it is well known that certain essential functions to the immune system decline with age, a situation which correlates with elevations in adrenal output of glucocorticoid steroid and abatement in production of other types of adrenal steroid hormones.

Excess glucocorticoid actions is widely believed to be associated with mood changes, depression, vertigo, memory loss or impairment, disorientation, and the like.

Elevated glucocorticoid action are also linked with hippocampal pathology in aging rodents. Basal plasma corticosterone levels in aged rats have been found to correlate with hippocampal atrophy and spatial learning deficits. It has also been found that cumulative exposure to constant high levels of glucocorticoids disrupts electrophysiological function, leading to atrophy and ultimately the death of hippocampal neurons. The compounds of Formula I and II are useful for preventing and treating hippocampal damage in a patient. Another aspect of the present invention is to prevent hippocampal damage in a patient comprising administering to said patient a prophylatically effective amount of a compound of Formula I or II. In another embodiment, the present invention is directed to a method of treating hippocampal damage in a patient, (e.g., reduce or ameliorate the effects of hippocampal damage and/or reduce or ameliorate hippocampal atropy) which comprises administering to the patient a therapeutically effective amount of a compound of Formula I or II.

It is widely believed that elevated glucocorticoid levels directly contribute to the development of cognitive impairments. Hippocampulatrophy has been reported in patients with Cushing's syndrome as a result of the hypersecretion of glucocorticoids. Thus, the compounds of Formula I and Formula II have an anti-glucocorticoid effect. They are useful in treating, ameliorating, preventing or retarding the progression of the unwanted condition or symptom or malady in a patient relating to an enhanced glucocorticoid effect, said method comprising administering to said patient an anti-glucocorticoid effected amount of a compound Formula I or II.

An enhanced glucocorticoid activity, as defined herein, refers to an enhanced glucocorticoid effect relative to normal which is attributable or results from various factors, such as hypersecretion of the glucocorticoid, enhanced activity of 11-beta-hydroxysteriod dehydrogenase, which is an enzyme which converts cortisone to cortisol the administration a glucocorticoid to patient, an enhanced concentration of glucocorticoid in the plasma relative to normal and the like. For example, the normal concentration of cortisol in the plasma in humans is about 7-20 ug/dL in the morning and about 3-13 ug/dL in the afternoon.

The compounds of the present invention are also useful in preventing or treating e.g., retarding immunosescence. Glucocorticoids, e.g., cortisol, are known to suppress the immune system and destroy lymphocytes in animals. As shown herein, the size of the thymus and the spleen are reduced in the presence of glucocorticoids, such as dexamethasone. The thymus and to some extent the spleen have a role in establishing the immunological capacity of the body. The thymus secretes hormones which are responsible for the production of cells with the capability of making antibodies and rejecting foreign bodies from the body. Moreover, both organs can produce lymphocytes and produce antibodies, which protect the body against invading microbes or foreign tissue. When the size of the thymus and spleen are reduced, their capacity to produce lymphocytes is also reduced, and the immune system is suppressed. Thus, as shown hereinabove, the compounds of the present protect against the atrophy of the spleen and thymus.

As one ages, the size of the spleen and the thymus also decreases. Further, as one ages, the cortisol levels also increase. Since glucocorticoids reduce the size of these two organs as one ages, the administration of compounds of Formula I and II retards the reduction of the size of these organs. Thus, the administration of the compounds of Formula I and II in antiglucocorticoid effective amounts retards the suppression of the immune system through the aging process.

It is also known that cortisol and other glucocorticoids damage and/or cause the atrophy of the hypothalmus, and more specifically causes hippocampalatrophy. (See, Lupien, et al., Nature Neuroscience, 1998, Vol. 1, 69-73). It is believed that mental disorders and spatial performance are associated with hippocampal function. Sustained glucocorticoid exposure damages the hippocampus in humans. Elevated glucocorticoid levels have been linked to the damage of the hippocampus and the impairment of learning and memory. As indicated hereinabove, as one ages, the amount of cortisol in the body increases. This memory loss as one ages is believed to be attributable to the increase in the cortisol concentration in the body. Thus, the administration of compounds I and II in antiglucocorticoid effective amounts retards the loss of memory. The compounds utilized in the present method are used in therapeutically effective amounts, i.e., in antiglucocorticoid effective amounts. These amounts are sufficient to detectably treat, ameliorate, prevent or detectably retard the progression of an unwanted condition or symptom associated with an excess concentration of glucocorticoids.

The compounds of Formula I and II in therapeutically effective amounts are useful to inhibit unwanted biological or cellular responses to glucocorticoid steroids, e.g., (1) glucocorticoid-induced immune suppression, (2) glucocorticoid-induced bone loss, or (3) modulation of glucocorticoid-induced gene transcription or expression, e.g., increased or decreased expression. The present invention includes administration of a therapeutically effective amount of the compound of Formula I or II to a subject having or being susceptible to developing a glucocorticoid-associated symptom or condition, wherein the condition or symptom is prevented, detectably ameliorated or its onset of progression is detectably delayed or slowed. Thus, the compounds of Formula I and II can be used to prevent or ameliorate, e.g., immune suppression, decreased immune cell proliferation or adverse neurological effects (e.g., mood changes, depression, memory loss or impairment, disorientation, headache, vertigo and the like) of glucocorticoid steroids.

An excess or unwanted level of glucocorticoid steroids (“GCS”) in a subject such as a mammal or a human can arise from natural causes, such as infections, cancer or injury, or such levels can arise from the use of GCS to treat various disease conditions or symptoms. Other causes of increased values of cortisol include: adrenal hyperplasia, adrenal adenoma, adrenal carcinoma, pituitary tumor, ectopic ACTH syndrome, pregnancy, prior exercise, prior tobacco smoking, emotional or physical stress, exogenous estrogens, chronic renal failure, hyperthyroidism, exogenous cortisone or hydrocortisone and the like.

The GCS that are associated with such conditions or symptoms can be natural or synthetic. GCS levels that are associated with or that cause an unwanted condition or symptom can arise from a natural disease or from the administration of a natural or synthetic glucocorticoid steroid to a subject such as a mammal, e.g., human. Thus, compounds of Formula I and II can be used diseases that are associated therewith for example. Moreover, corticosteroids are used to treat the following disorders: Achilles tendon disorders, Addison's disease, ankylosing spondylitis, asthma, athletic injury, atopic dermatitis, bacterial meningitis, carcinoid tumor, chickenpox, chronic lymphocytic leukemia, congenital adrenal hyperplasia, COPD, Crohn's disease, croup, cystic fibrosis, discoid lupus erythematosus, focal segmental glomerulosclerosis, gout, hay fever, Henoch-Schonlein purpura, hypercalcemia, idiopathic hypereosinophilic syndrome, idiopathic thrombocytopenic purpura, infectious mononucleosis lichen planus, minimal change disease, multiple myeloma, multiple schlerosis, neutropenia, nummular dermatitis, pemphigus, polyarteritis nodosa, polymyositis, psoriasis, rapidly progressive glomerulonephritis, recurrent aphthous stomatitis, respiratory failure, rheumatoid arthritis, sarcoidosis, spinal cord injury, systemic lupus erythematosus, tendenitis, toxic epodermal necrolysis, transplantation, tuberculosis, typhoid fever, ulcerative colitis and furthermore, Cortisol is used to treat the following disorders: Addison's disease, Cushing's disease, ectopic ACTH syndrome, hyponatremia, liver disease, pediatric cardiopulmonary resuscitation. The compounds of Formula I or II thus can limit the unwanted side effects of GCS, without eliminating all of their beneficial, e.g., anti-inflammatory, effects. Thus, in some embodiments, a therapeutic treatment using a compound of Formula I and II is coadministered with one or more GCS. The GCS are used in a number of clinical situations, e.g., in chemotherapy, to decrease the intensity or frequency of flares or episodes of inflammation or autoimmune reactions in conditions such as rheumatoid arthritis, osteoarthritis, ulcerative colitis, bronchial asthma, psoriasis or systemic lupus erythematosus. Other side effects include but are not limited to, asceptic necrosis host defense alterations opportunistic infections and the like. The compounds of Formula I and II reduce the side effects associated with the glucocorticoid treatment of these indications such as endocrine disorders, including adrenal cortical insufficiency, congenital adrenal hyperplasia, nonsuppurative thyroiditis, hypercalcemia associated with cancer, rheumatic disorders, including psoriatic arthritis, rheumatoid arthritis, ankylosing spondylitis, bursitis, acute nonspecific tenosynovitis, acute gouty arthritis, post-traumatic osteoarthritis, synovitis of osteoarthritis, epicondylitis, collagen diseases, including systemic lupus erythematosus, acute rheumatic carditis, dermatological diseases, including pemphigus, bullous dermatitis herpetiformis, severe erythema multiforme, exfoliative dermatitis, mycoses fungicides, severe psoriasis, severe seborrheic dermatitis, allergic states, including allergic rhinitis, bronchial asthma, contact dermatitis, atopic dermatitis, serum sickness, drug hypersensitivity reactions, ophthalmic diseases, including allergic conjunctivitis, keratitis, allergic corneal marginal ulcers, herpes zoster ophthalmicus, iritis and iridocyclitis, chorioretinitis, anterior segment inflammation, diffuse posterior uveitis and chorioditis, optic neuritis, sympathetic ophthalmia, respiratory diseases, including symptomatic sarcoidosis, Loeffler's syndrome, Berylliosis, pulmonary tuberculosis, aspiration pneumonitis, hematological disorders, including idopathic and secondary thrombocytopenic purpura, acquired hemolytic anemia, erythroblastopenia, congenital hypoplastic anemia, neoplastic diseases, including leukemias and lymphomas, edematous states, gastrointestinal diseases, including ulcerative colitis, regional enteritis, cerebral edema, including brain tumor, craniotomy, head injury, aging, and the like.

Adverse reactions that would be ameliorated by compounds of Formula I or II either through direct action or through allowing a lower dose of glucocorticoid to be used, for example: include but are not limited to fluid and electrolyte disturbances, including sodium retention, fluid retention, congestive heart failure, potassium loss, hypokalemic alkalosis, hypertension, muskuloskeletal, including muscle weakness, steroid myopathy, loss of muscle mass, osteoporosis, vertebral compression fractures, asceptic necrosis, pathologic fracture of long bones, tendon rupture, gastrointestinal, including peptic ulcer, perforation of small and/or large bowel, pancreatitis, abdominal distention, ulcerative esophagitis, dermatologic, including impaired wound healing, thin fragile skin, petechiae and ecchymoses, erythema, increased sweating, suppressed reactions to skin tests, allergic dermatitis, urticaria, angioneurotic edema, neurologic, including convulsions, intracranial pressure, vertigo, headache, psychic disturbances, endocrine, including menstrual irregularities, cushingoid state, suppression of growth in children, adrenocortical and or pituitary unresponsiveness, decreased carbohydrate tolerance, lanifestatio's of latent diabetes meilitus, increased requiremenis or insulin or oral hypoglycemic agents in diabetics, hirsutism, ophthalmic, including posterior subcapsular cataracts, increased intraocular pressure, glaucoma, exophthalmus, metabolic, including negative nitrogen balance, cadiovascular, including myocardial rupture, other, including hypersensitivity, thromboembolism, weight gain, increased appetite, nausea, malaise, hiccups, nightmares, hallucinations, immune deficiencies, and the like.

The compounds of Formula I and II are useful to counteract the adverse effects or toxicities of glucocorticoids without negating all of the desired therapeutic capacity of the glucocorticoids. This allows the continued use, or a modified dosage of the glucocorticoid, e.g., an increased dosage, without an intensification of the side effects or toxicities or a decreased glucocorticoid dosage. T he side-effects or toxicities that can be treated, prevented, ameliorated or reduced include one or more of the following: bone loss, reduced bone growth, enhanced bone resorption, osteoporosis, immunosuppression, increased susceptibility to infection, mood or personality changes, depression, headache, vertigo, high blood pressure or hypertension, muscle weakness, fatigue, nausea, malaise, peptic ulcers, pancreatitis, thin or fragile skin, growth suppression in children or preadult subjects, thromboembolism, cataracts, and edema.

In another embodiment, the compounds of Formula I or II or combination thereof can be used in combination with a statin for treating or preventing any of the diseases, maladies conditions or disorders described herein. As defined herein, a statin is a HMG-CoA-reductase inhibitor that inhibits HMG-CoA reductase. Thus, the statins are compounds having action of lowering blood cholesterol levels by inhibiting 3-hydroxy-3-methylglutaryl CoA (HMG-CoA) reductase. They have the advantages of lowering LDL. A large number of naturally or synthetically obtained or synthetically modified compounds have been found to inhibit HMG-CoA reductase. These compounds form a category of agents useful for practicing the present invention. Traditionally these agents have been used to treat individuals with hypercholesterolemia. Examples include statins, which are commercially available, such as lovastatin and mevinolin disclosed in U.S. Pat. No. 4,231,938, pravastatin and pravastatin sodium disclosed in U.S. Pat. No. 4,46,227, fluvastatin and fluvastatin sodium and XU 62-320 disclosed in EP 0 114 027 and U.S. Pat. No. 4,739,073, atorvastatin disclosed in U.S. Pat. No. 5,273,995, itavastatin also known as NK-104 disclosed in EP304063, mevastatin disclosed in U.S. Pat. No. 3,983,140, rosuvastatin, velostatin and synvinolin and simvastatin disclosed in U.S. Pat. Nos. 4,448,784 and 4,450,171, cerivastatin and numerous others described in U.S. Pat. Nos. 5,622,985, 5,135,935, 5,356,896, 4,920,109, 5,286,895, 5,262,435, 5,260,332, 5,317,031, 5,283,256, 5,256,689, 5,182,298, 5,369,125, 5,302,604, 5,166,171, 5,202,327, 5,276,021, 5,196,440, 5,091,386, 5,091,378, 4,904,646, 5,385,932, 5,250,435, 5,132,312, 5,130,306, 5,116,870, 5,112,857, 5,102,911, 5,098,931, 5,081,136, 5,025,000, 5,021,453, 5,017,716, 5,001,144, 5,001,128, 4,997,837, 4,996,234, 4,994,494, 4,992,429, 4,970,231, 4,968,693, 4,963,538, 4,957,940, 4,950,675, 4,946,864, 4,946,860, 4,940,800, 4,940,727, 4,939,143, 4,929,620, 4,923,861, 4,906,657, 4,906,624, RE36,520, and U.S. Pat. No. 4,897,402, the disclosures all of which patents are incorporated herein by reference.

The preferred statin is selected from the group consisting of lovastatin, pravastatin, simvastatin, atorvastatin, rosuvastatin, fluvastatin, itavastatin, and cerivastatin. The statin may be present in any amount; however, it is preferably present in therapeutic effective amounts, especially amounts effective to lower cholesteral levels. The statin can be present in dosages of 0.05 mg to 100 mg. If the statin is lovastatin, it is preferably present in the range of 2 mg to 50 mg; if the statin is pravastatin, it is preferably present in the range of 2 mg to 50 mg; if the statin is simvastatin, it is preferably present in the range of 2 mg to 100 mg; if the statin is atorvastatin, it is preferably present in the range of 2 mg to 100 mg; if the statin is rosuvastatin, it is preferably present in the range of 2 mg to 100 mg; if the statin is fluvastatin, it is preferably present in the range of 2 mg to 50 mg; if the statin is itavastatin it is preferably present in the range of 0.2 mg to 100 mg; and if the statin is cerivastatin it is preferably present in the range of 0.05 mg to 2 mg.

It is to be noted that the amount of a given statin in a dosage form of this invention can be the same as the amount of that statin in currently available dosage forms of that statin alone or it can be an amount that is lower than the amount of that statin in currently available dosage forms of that statin alone. The presence of the statin augments or supplements the effect of compounds of Formula I or II.

Unless indicated to the contrary, the preferred amounts given hereinabove are also the preferred amounts for treating or preventing the conditions, diseases or maladies discussed herein.

The following non-limiting examples further illustrate the present invention.

### EXAMPLE 1

Twelve patients were treated with 16α-fluoro-5-androstene-17-one.

Since excessive alcohol consumption or increased caloric intake elevates serum triglycerides, for one week, prior to the start of the study, the subjects were not allowed to ingest excessive amounts of coffee, tea or alcohol or to follow a diet which deviated notably from the normal diet. The amounts did not exceed 0.5 L of beer or 0.25L of wine per day; drinking up to 4 cups of xanthine containing beverages per day was allowed. Smoking and intake of xanthine-containing beverages or food (coffee, tea, cola, chocolate) and alcohol within 24 hours, before and during the stay in the clinical research facilities were not allowed.

The human patients received 1600 mg of 16α-fluoro-5-androstene-17-one daily. Controls were set up so that they received 1600 mg of placebo. The triglyceride concentration in the plasma were measured pretreatment and at the end of the study. There was a significant triglyceride-lowering effect caused by the drug in the 14-day, multiple dose, study.

In 12 patients (excluding 2 placebos) receiving 1600 mg of 16α-fluoro-5-androsten-17-one daily, 4 patients, 3 males and one female, had pretreatment triglyceride levels>200 mg/dL. In this group of 4 patients there was a highly significant reduction in triglyceride values, whereas there was no apparent effect in the 8 patients with non-elevated triglycerides. The decline in one female patient was additionally quite striking. The data in the one female, 3 males, and the combined data are shown below.

Conc. (mg/dL)

Elevated Triglyceride Group

| | |
|---|---|
| Female (1) | |
| Pretreatment | 540 ± 42 (n = 2) |
| Treatment | 118 ± 19 (n = 3) |
| | p˜0.001 |
| Male (3) | |
| Pretreatment | 333 ± 75 (n = 6) |
| Treatment | 168 ± 61 (n = 8) |
| | p˜0.001 |
| Female and Male (4) | |
| Pretreatment | 385 ± 116 (n = 8) |
| Treatment | 154 ± 57 (n = 11) |
| | p˜0.0001 |

Normal Triglyceride Group

| | |
|---|---|
| Pretreatment | 106 ± 34 (n = 16) |
| Treatment | 128 ± 77 (n = 23) |

In addition, the fasting plasma glucose (FPG) levels were also measured in the patients before and after treatment. The results are as follows. In the study, 3 of the 4 patients with elevated triglycerides had pre-treatment FPG levels>6.1 mM. The American Diabetes Association (ADA) has recently classified individuals with FPG of 6.1-6.9 mM as having impaired fasting glucose (IFG). Such individuals have 40% probability of developing ADA criteria for diabetes within 5 years.

FPG LEVELS OF INDIVIDUALS WITH IFG

| Pretreatment | Treatment |
|---|---|
| 6.28 ± 0.33 (6) | 5.63 ± 0.45 (8) (p < 0.02) |

There was a significant lowering of FPG levels during treatment using 16α-fluoro-5-androsten-17-one. These data suggest that at least part of the triglyceride-lowering effect of the compounds described herein is attributable to improvement in insulin sensitivity.

### EXAMPLE 2

Five hypertriglyceridemic patients had high baseline HDL levels (>50 mg/dL, 64.2±8.5, n=10, mean±S.D.) and four hypertriglyceridemic patients had low HDL levels 40.4±4.1, n=8). They were treated as before, as described in Example 1, except they received 1200 mg of 16α-fluoro-5-androsten-17-one daily. Controls were set up so that they received 1200 mg of placebo. The triglyceride concentration in the plasma were measured pretreatment and at the end of the study. The results are as follows.

In those patients with high HDL levels, the triglycerides fell from 330±163 (n=10) to 214±56.1 (n=5). However, in 4 patients with low HDL levels (40.0±4.1), treatment with 1200 mg of 16α-fluoro-5-androsten-17-one daily for 3 to 12 weeks lowered triglyceride levels from 245±55.1 (n=8) to 139±10.6 (n=4), p<0.01. Thus, these experiments show that this compound is effective in lowering plasma triglycerides in patients with high triglycerides and low HDL.

It is to be noted that the lipid profile of high triglyceride (>200 mg/dL) and low HDL (<40 mg/dL if male and <45 mg/dL if female) is associated with a high cardiovascular disease mortality. This was demonstrated in a study in the Lipid Research Clinic Study on 1405 middle-aged women. See Bass, et al. Arch. Int. Med., 153:2209, 1993. In the Copenhagen Male Study in 2910 middle-aged men, individuals in the highest third of triglyceride level (avg:248 mg/dL) and lowest third of HDL (avg:39 mg/dL) experienced an increased incidence of ischemic heart disease that was at least as great as individuals with isolated high LDL (highest one-fifth of LDL level) (See, Teppesen, et al., Atherioscler. Throm. Vase. Biol., 17:1114 (1997). The high-to-low HDL lipid profile is the characteristic of dyslipidemia associated with insulin resistance.

### EXAMPLE 3

The following example, tested the effects of buccal administration of 16α-fluoro-5-andorsten-17-one (hereinafter “drug”).

Eight-week-old male BKS.Cg-m⁺/₊Lepr^{db} mice were obtained from Jackson Laboratories. The mice were initially housed five per cage on Alphacel bedding with ad libitum access to Purina 5015 chow and acidified water. The mice wee housed in the Central Animal Facility (6^{th} Floor, Pharmacy building) with twelve hours of alternating light and darkness.

Five days later, the chow was removed from the mice at approximately 3:30 p.m. This was done so that a fasting plasma glucose level could be obtained on the next day. The next day, a pretreatment plasma glucose determination was made. The mice were lightly anesthetized with Isoflurane and were bled form the orbital sinus (˜400 μL of blood was taken). The blood was obtained between 10:00 a.m. and noon. Blood was kept on ice until analysis. After the blood for the glucose measurement was taken out, the remaining blood was centrifuged at 3000×g for 15 minutes. The plasma was removed and frozen for use in determining plasma triglyceride levels.

A. Fasting Plasma Triglyceride Determination:

Plasma triglyceride levels were determined with a Sigma kit (334-UV). The determination is based on the enzymatic hydrolysis of triglycerides to glycerol and free fatty acids by lipase. Glycerol was subsequently phosphorylated by ATP to produce glycerol-I-phosphate and ADP. ATP was regenerated by a pyruvate kinase-catalyzed reaction between ADP lactate with simultaneous oxidation of equimolar amounts of NADH in the presence of lactate dehydrogenase. NADH absorbs at 340 nm, thus the decrease in absorbance, measured at this wavelength, is directly proportional to the triglyceride concentration in this sample.

Triglyceride Reagent A (containing ATP, lactate dehydrogenase, lipase, NADH, phosphoenol pyruvate, pyruvate kinase) and Triglyceride Reagent B were reconstituted with 10 mL and 2 mL of distilled water respectively. The Sample Reagent was prepared by adding 0.25 mL of Triglyceride reagent B to 10 mL to Triglyceride Reagent A. Blank, standard (50, 100 and 200 mg/dL) and sample tubes were prepared. One mL of Sample Reagent was added to all tubes. To the Blank tube, 20 μL of distilled water was added. For each Sample tube, 20 μL of plasma was added to the tube. All tubes were incubated at room temperature for 10 minutes. The tubes were read against a Reference containing distilled water. Triglyceride concentration in the samples were calculated by subtracting the absorbance of the Sample form the absorbance of the Blank and using the following formula:

Serum triglyceride concentration=ΔA

B. Treatment of Mice:

One week after obtaining the mice, the mice were distributed into either groups of 6 mice (Control, buccal and 5 mg/kg drug buccal), or 7 mice (10 mg/kg drug buccal). The mice were weighed.

The buccally administered groups were treated with a suspension of drug, 16α-5-androsten-17-one obtained from Eminent Services Corp. The suspension consisted of 100 mg/mL of micronized fluasterone (16α-fluoro-5-androsten-17-one) in 0.9% saline plus 2% Tween 80 and 0.4% carboxymethyl cellulose. The mice were lightly anesthetized with isoflurane and then injected i.m. with 0.05 mL of a solution of ketamine (50 mg/kg), xylazine (10 mg.kg) and atropine (0.1 mg/kg) injected near tail). Approximately 10 minutes post injection, while the animals were anesthetized, the mice were placed on their backs and were treated with one-half of the dose of drug necessary in each buccal area. The mice remained anesthetized and on their backs for approximately 30 minutes post-treatment. The 5 mg/kg drug group was treated with 2.04 μL of suspension (1.02 μL per buccal area) and the 10 mg/kg drug group was treated with 4.2 μL(2.1 μL per buccal area) while the Control Buccal group received 4.2 μL (2.1 μL per buccal area) for the duration of the experiment. The cages of the mice were placed on heating pads on a low temperature to prevent loss of animals due to hypothermia during anesthesia. The cages were kept on the heating pads for 30 minutes after all the mice in the cage were awake.

During the second week of the experiment, the mice in the ten mg/kg buccal group were coming out of the anesthesia quicker than the 5 mg/kg group and Control groups (20 minutes versus 30 minutes).

The results are tabulated hereinbelow.

| | | |
|---|---|---|
| | 5 mg/kg | 10 mg/kg |
| | buccal | buccal |
| Control, buccal | drug Plasma | drug Plasma |
| Plasma | triglycerides | triglycerides |
| triglycerides mg/dl | mg/dl | mg/dl |

(Pre-treatment)

| | | | |
|---|---|---|---|
| Mice 1 | 123.8 | 110.7 | 93.7 |
| Mice 2 | 161.1 | 153.8 | 102.1 |
| Mice 3 | 126.1 | 105.9 | 162.1 |
| Mice 4 | 115.6 | 116.8 | 97.1 |
| Mice 5 | 159.4 | 222.8 | 102.1 |
| Mice 6 | 117.8 | 120.6 | 89.4 |
| Mice 7 | | | 184.4 |
| | 134.0 ± 20.7 | 138.4 ± 44.6 | 118.7 ± 38.1 |

(AFTER 6 DAYS OF TREATMENT)

| | | | |
|---|---|---|---|
| Mice 1 | 126.4 | 77.2 | 108.7 |
| Mice 2 | 156.8 | 106.0 | 83.5 |
| Mice 3 | 165.0 | 87.3 | 96.8 |
| Mice 4 | 156.2 | 414.0 | 61.2 |
| Mice 5 | 179.8 | 146.6 | 104.6 |
| Mice 6 | 157.8 | 105.4 | 64.3 |
| Mice 7 | | | 156.7 |
| | 157.0 ± 17.4 | 110.6 ± 28.0 | 96.5 ± 32.4 |
| | | p < 0.01 | p < 0.01 |
| | | vs. control | vs. Control |
| | | buccal | buccal |

(AFTER 13 DAYS OF TREATMENT)

| | | | |
|---|---|---|---|
| Mice 1 | 132.3 | 97.8 | 102.0 |
| Mice 2 | 172.0 | 89.3 | 83.5 |
| Mice 3 | 154.6 | 65.3 | 93.6 |
| Mice 4 | 199.9 | 79.1 | 133.5 |
| Mice 5 | 193.0 | 78.3 | 118.1 |
| Mice 6 | 151.7 | | 97.8 |
| Mice 7 | | | 99.2 |
| | 167.3 ± 26.0 | 82.0 ± 12.3 | 104.0 ± 16.6 |
| | | p < 0.0005 | P < 0.001 |
| | | vs. Control | vs. Control |
| | | buccal | buccal |

The data herein clearly show that buccally administered 16α-fluoro-5-andrsten-17-one, even at concentrations as low as 5 mg/kg and 10 mg/kg significantly lower the triglyceride levels in mice.

### EXAMPLE 4

Female CD-1 mice were obtained from Charles River Laboratories, Kingston, N.Y. at 43-45 days of age. The mice were housed five per cage in plastic shoebox cages on corn cob bedding in the Fels Animal Facility at 72°±2° F. with 50%±5% humidity and twelve hours of alternate light and darkness. The mice had ad libitum access to Purina chain 5015 and acidified water for pretreatment.

Six days later, the mice were weighed, earmarked and redistributed into six groups for treatment: 1) a control group, 2) a group treated with dexamethasone (“DEX”), a glucocorticoid which induces thymic and splenic atrophy, 3) a group treated with 200 mg/kg 16α-fluoro-5-androsten-17-one (hereinafter “drug”), 4) a group treated with 400 mg/kg of, 5) a group treated with dexamethasone and 200 mg/kg drug and 6) a group treated with dexamethasone and 400 mg/kg drug. The treatment in the groups were as follows and were conducted simuitaneously.

The control group was intubated with 0.2 mL of sesame oil for three days. On the third day after commencement of the intubation, the mice were injected subcutaneously with 0.05 mL of absolute ethanol one hour after the last treatment with sesame oil.

The second group was intubated with 0.2 mL of sesame oil for three days. On the third day after the commencement of the intubation, the mice were injected subcutaneously with 1.6 mg of dexamethasone dissolved in 0.05 mL of absolute ethanol approximately one hour after the last treatment with sesame oil.

The third and fourth groups were intubated with 200 mg/Kg and 400 mg/Kg drug, respectively, suspended in 0.2 mL of sesame oil for three days. After commencement of the intubation, the mice were injected subcutaneously with 0.05 mL of absolute ethanol approximately one hour after the last treatment with sesame oil.

The fifth and sixth groups were intubated with 200 mg/kg and 400 mg/kg, respectively, of drug, suspended in 0.2 mL of sesame oil for three days. On the third day after commencement of the intubation, the mice were injected subcutaneously with 1.6 mg of dexamethasome dissolved in 0.05 mL of absolute ethanol approximately one hour after the last treatment with sesame oil.

All of the mice were sacrificed by an overdose of CO₂ approximately 24 hours after the injection of ethanol (first, third and fourth groups) or approximately 24 hours after injection with dexamethasone (second, fifth and sixth groups). The mice were weighed and the thymus was excised, cleaned of adventia, rinsed in phosphate-buffered saline (P.B.S.), blotted and weighed. The spleen was also excised, cleaned, rinsed, blotted and weighed.

The results are tabulated hereinbelow:

TABLE 1

| | Body Weight | Thymus Wt. | | |
|---|---|---|---|---|
| Treatment | (gm) | (mg) | Spleen Wt. (mg) | No. |
| Control | 24.0 ± 1.0 | 67.6 ± 8.1 | 100.6 ± 13.8 | 10 |
| DEX | 23.4 ± 1.1 | 36.8 ± 7.1 | 45.2 ± 7.5 | 10 |
| 200 mg/kg | 23.7 ± 0.9 | 65.0 ± 5.8 | 103.5 ± 9.2 | 10 |
| drug | | | | |
| 400 mg/kg | 24.7 ± 1.9 | 74.3 ± 7.1 | 129.4 ± 17.9 | 10 |
| drug | | | | |
| 200 mg/kg | 23.6 ± 1.3 | 39.5 ± 9.1 | 55.9 ± 19.7 | 9 |
| drug + DEX | | | | |
| 400 mg/kg | 23.7 ± 1.1 | 53.6 ± 5.7* | 69.0 ± 5.8* | 10 |
| drug + DEX | | | | |

*Significantly greater than DEX group, p < 0.001.

As shown in Table 1, treatment with 16α-fluoro-5-androstene-17-one at 400 mg/kg significantly reduced dexamethasone-induced thymic and splenic atrophy. The data clearly show that 16α-fluoro-5-androsten-17-one produces an anti-glucocorticoid effect.

In addition, the data show that the drug prevents the dexamethasone, a glucocorticoid, from reducing the size of the spleen and the thymus.

### EXAMPLE 5

HDL-Lowering Effect of Oral Fluasterone

(a) In a phase I trial in normal young men and women fluasterone was administered in a daily oral dose of 800 mg or 1600 mg for 14 days. The effect of this treatment on HDL levels in males and females is seen in the table hereinbelow.

HDL Levels (mg/dL) - 800 mg Fluasterone

| | Males (6) | Females (6) |
|---|---|---|
| Pretreatment | 50.3 ± 20.1 | 59.2 ± 7.0 |
| Treatment | 45.3 ± 15.9 | 46.1 ± 6.2 |
| | | (less than pretreatment) |
| | | (p < 0.015) |

HDL Levels (mg/dL) - 1600 mg Fluasterone

| | Males (7) | Females (5) |
|---|---|---|
| Pretreatment | 46.1 ± 6.6 | 45.3 ± 10.1 |
| Treatment | 40.2 ± 7.4 | 43.0 ± 8.1 |

(b) Trial in Arthritis Patients

In this trial patients with rheumatoid arthritis were treated for 3 to 12 weeks with 1200 mg oral fluasterone daily. A placebo group was also included.

HDL Levels

| | | |
|---|---|---|
| Males | Females | Combined |

Fluasterone

| | | | |
|---|---|---|---|
| Pretreatment | 47.0 ± 9.1 (4) | 59.8 ± 14.6 (16) | 57.4 ± 14.7 |
| | | | (20) |
| Treatment | 47.0 ± 5.8 (4) | 46.8 ± 9.6 | 46.9 ± 8.9 |
| | | (p < 0.01) | (p < 0.02) |

Placebo

| | | | |
|---|---|---|---|
| Pretreatment | 33.5 (1) | 56.5 ± 21.8 (4) | 51.9 ± 21.5 |
| | | | (5) |
| Treatment | 35 | 53.0 ± 18.1 | 49.4 ± 17.6 |

(c) Trial in Syndrome X Patients

Eleven patients were enrolled in the trial. The average age was 50.2±12.8 years, and there were 10 males and 1 female patient enrolled. Body weight and body mass index were 87.8±17.9 kg and 27.7±3.0 kg/m², respectively. All patients carried a diagnosis of dyslipidemia, including elevated triglycerides, for an average of 11.7±6.4 years.

In this randomized, double-blind, crossover study, the 11 patients underwent eligibility screening (physical exam, medical history, laboratory evaluation, PSA, TSH), followed by a six-week lead-in phase (study weeks 1-6) that included diet (AHA Step II), and discontinuation of lipid-lowering therapy. At week 4, qualifying lipid testing was performed, followed by randomization to study treatment two weeks later. Four-weeks of each of the two study treatments, fluasterone 600 mg twice daily or placebo, were then administered (weeks 7 thru 10 and 15 thru 18), separated by a 4-week washout period. At baseline, and at the end of each treatment period measurement of total serum cholesterol, triglycerides, apolipoprotein A1, and the cholesterol and triglyceride content of VLDL, LDL, and HDL was done.

The baseline triglyceride level (mg/dL) was 509±184 and the baseline HDL level (mg/dL) was 33.0±3.5.

Comparing fluasterone with placebo, fluasterone significantly lowered triglyceride levels (−165±76 vs −22.6±74, least square means±S.E.M., p<0.005) and also apparently lowered HDL levels (−5.8±4.1 vs 9.3±6.8, p=0.068).

The results of the above three trials demonstrate an HDL-lowering effect with high dose oral fluasterone therapy. However, since the risk of atherosclerosis is inversely proportional to the levels of HDL, it would be highly desirable to eliminate, or greatly reduce, the HDL-lowering effect seen with oral fluasterone therapy. This is achieved if given buccally or subcutaneously.

### EXAMPLE 6

In this clinical trial oral fluasterone (1200 mg) was administered daily to patients with rheumatoid arthritis for 3 to 12 weeks. Nine patients had baseline triglyceride levels 200 mg/dL. These patients were divided into obese (BMI>30) and non-obese (BMI<30) groups. One patient with a baseline of 176 mg/dL was included in the obese group, since this patient had a very high BMI (42.9). However, the conclusion would be the same without the addition of this patient.

Triglycerides (mean ± S.D.)

| | Baseline | Treatment |
|---|---|---|
| Obese | 231 ± 57.1 (10) | 135 ± 12.1 (5) |
| Non-obese | 330 ± 163 (10) | 248 ± 89.8 (5) |

The baseline levels was the mean value of week −2, 0 and the treatment levels were the last values obtained (week 3 to 12) for each patient. The obese patients has a BMI of 36.6±4.0 and the non-obese a BMI of 27.1±1.5. The obese patients experienced a significantly greater decline in triglyceride level than the non-obese (p=0.0113, group effect) as well as individually at week 3, 6, and 9 (p<0.041).

The greater decline in triglyceride levels was observed in the obese patients notwithstanding their lower baseline level (232 vs. 310), and since the percent decline in baseline in both the obese and non-obese patients was greater the higher the baseline, this strengthens the conclusion.

Since obese patients with endogenous hypertriglyceridemia have significantly higher plasma insulin levels than non-obese patients with endogenous hypertriglyceridemia, this study indicates that fluasterone produces an anti-hypertriglyceridemic effect, at least in part, by improving insulin sensitivity.

### EXAMPLE 7

The study was a phase I/II randomized, double blind, dose-ranging, placebo-controlled, evaluation of the safety, tolerance, and activity on lipid laboratory markers after transmucosal (buccal) administration of two dose levels (20 mg or 80 mg) of fluasterone (16α-fluoro-5-androsten-17-one) compared to placebo in patients with Metabolic Syndrome. Study treatment consisted of eight weeks of buccal tablet administration of fluasterone or placebo control and subsequent follow-up for two weeks.

After patient consent had been obtained, patients were screened for eligibility into the study. Following informed consent, screening and laboratory testing, patients entered a six-week dietary run-in phase during which they followed a Step II AHA diet, and discontinued all lipid-lowering agents. At 4 weeks into the dietary run-in phase, qualifying lipid and endocrine laboratory measurements were performed. After the run-in phase, patients were randomized to receive either fluasterone or placebo control. Patients received fluasterone or placebo daily for 8 weeks. Patients were followed for an additional 2 weeks for total study duration of 16 weeks. The dose levels evaluated in this study were either 20 mg or 80 mg buccal tablets of fluasterone or placebo control that were formulated to dissolve between the lower gingival surfaces of the jaw and buccal mucosa of the cheek pouch.

Administration of Study Medicine

Fluasterone or placebo equivalent was administered as either one or four buccal tablets. Each buccal tablet contained 16-α-fluoro-5-androsten-17-one, mannitol, crospovidone, magnesium stearate, polyethylene glycol 3350, silica gel and sodium lauryl sulfate. Patients were instructed to ingest water prior to placing the buccal tablets in their mouth. The tablets were held in the mouth without disturbance for approximately 10 minutes. Patients were instructed not to chew or swallow the tablets and not drink or eat for 30 minutes after study drug administration.

Duration

The study consisted of a 6-week dietary run-in phase during which they followed a Step II AHA diet and discontinued all lipid-lowering agents. After the run-in phase, patients who qualified based on lipid and endocrine laboratory measurements at the qualifying visit (week −2) were randomized to receive fluasterone or placebo control. Patients received fluasterone or placebo control daily for 8 weeks. Patients were followed for an additional 2 weeks to collect adverse events and an additional lipid profile for total study duration of 16 weeks.

Blood samples were collected for lipid determinations at the qualifying visit (week −2), at study day 1, and at weeks 2, 4, 6, 8, and 10.

There were 8 patients treated with 80 mg fluasterone, 9 with 20 mg fluasterone, 3 patients with 80 mg placebo, and 4 patients with 20 mg placebo.

The results are tabulated hereinbelow:

Effect of Fluasterone on Triglyceride and HDL Levels

| | Triglycerides | HDL |
|---|---|---|
| | (mg/dL) | (mg/dL) |
| Fluasterone - 80 mg | | |
| Baseline (−2, 0) | 455 ± 157 (16) | 40.8 ± 8.9 (16) |
| Week 2, 4, 6, & 8 | 342 ± 111 (32) | 41.1 ± 8.1 (32) |
| Week 8 | 292 ± 92.4 (8) | 41.6 ± 9.4 (8) |
| Fluasterone - 20 mg | | |
| Baseline (−2, 0) | 507 ± 192 (18) | 31.6 ± 6.2 (18) |
| Week 2, 4, 6, & 8 | 523 ± 311 (36) | 32.6 ± 6.4 (36) |
| Week 8 | 538 ± 285 (9) | 33.3 ± 6.5 (9) |
| Combined Placebo | | |
| Baseline (−2, 0) | 411 ± 127 (14) | 37.0 ± 8.1 (14) |
| Week 2, 4, 6, & 8 | 404 ± 180 (28) | 38.4 ± 8.3 (28) |
| Week 8 | 389 ± 176 (7) | 38.9 ± 8.0 (7) |

As shown in the Table, treatment of patients with 80 mg buccal fluasterone reduced the mean (week 2, 4, 6, & 8) triglyceride level by at least 25% compared to baseline andthe mean 8-week level by 36% compared to baseline.

The 80 mg fluasterone buccal dose was effective. Moreover, unlike the oral dose the buccal dose produced no apparent decline in HDL level.

The lack of an HDL-lowering effect with the buccal formulation is consistent with the following data in Example 8 indicating that first-pass hepatic metabolism greatly enhances fluasterone androgenicity. The data shown that a buccal formulation of fluasterone can be used to eliminate or greatly reduce androgenicity while retaining efficacy.

### EXAMPLE 8

Five out of eight patients in the 80-mg buccal fluasterone group had a BMI. 30 and five out of seven patients in the combined placebo group had a BMI 30. The triglyceride level in the 80 mg fluasterone group declined from baseline by 34% at week 2, 4, 6, and 8 and by 35% at week 8. In the combined placebo group at the corresponding time points the triglyceride levels increased by 6% and 7% respectively. In this subset of obese patients, the decline in triglyceride level from baseline in the 80 mg buccal fluasterone group was significantly greater than that in the combined placebo group (p=0.0368). There was also no decline in HDL levels from baseline in the fluasterone obese group as was observed in the combined obese and non-obese patients as shown in Example 7.

Effect of Buccal Fluasterone in Obese Subset

| | Triglycerides | HDL |
|---|---|---|
| | (mg/dL) | (mg/dL) |
| Fluasterone - 80 mg | | |
| Baseline (−2, 0) | 472 ± 169 (10) | 39.2 ± 7.3 (10) |
| Week 2, 4, 6, and 8 | 313 ± 93.7 (20) | 40.3 ± 6.3 (20) |
| Week 8 | 307 ± 106 (5) | 40.3 ± 4.6 (5) |
| Combined Placebo | | |
| Baseline (−2, 0) | 395 ± 145 (10) | 33.9 ± 3.9 (10) |
| Week 2, 4, 6, and 8 | 419 ± 203 (20) | 34.2 ± 3.5 (20) |
| Week 8 | 424 ± 202 (5) | 35.0 ± 3.9 (5) |

The finding that 80 mg fluasterone administered by the buccal route appears to lower triglycerides more effectively in the obese patients vs. the non-obese is consistent with the data in Example 14 demonstrating a significantly greater triglyceride-lowering effect of 1200 mg oral fluasterone in obese vs. non-obese arthritis patients. These data suggest that fluasterone reduces triglyceride levels, at least partly, by improving insulin sensitivity.

### EXAMPLE 9

Eight week old male BKS.Cg-m⁺/₊Lepr^{db} mice were obtained from Jackson Laboratories. These mice have a point mutation in the leptin receptor gene and produce a truncated receptor with impaired signaling capacity. These animals are severely insulin resistant and manifest an uncontrolled rise in blood sugar, severe depletion of the insulin-producing beta-cells of the pancreatic islets, and death by 10 months of age. The male animals also have very low plasma testosterone levels and as a consequence undeveloped seminal vesicle glands. Administration of androgens to these mice produces an increase in seminal vesicle weights similar to that seen in castrated males. Thus these animals can be used to assess androgenic potency of various compounds.

The mice were housed five per cage on Alphacel bedding with ad libitum access to Purina 5015 chow and acidified water. The mice were housed in the Central Animal Facility (6^{th} floor, Pharmacy building) with twelve hours of alternating light and darkness.

A week later, the mice were treated subcutaneously (s.c.) with 0.05 mL fluasterone (16α-fluoro-5-androsten-17-one) suspended in an Emulphor-saline vehicle to give either a dose of 5 mg/kg s.c. or 20 mg/kg s.c. or intraperitoneally (i.p.) with 0.05 mL of fluasterone suspended in an Emulphor-saline vehicle to give a dose of 5 mg/kg. The Control mice received the Emulphor-saline vehicle s.c.

All the mice were anesthetized lightly with isoflurane before treatment with fluasterone to minimize the risk of stressing the mice while giving the s.c. dose. Their food was removed the afternoon before administration with drug and the chow was to the mice the next morning immediately after treatment. All the mice had their chow removed daily and restored just after fluasterone administration. During the week, the mice had access to chow for approximately 6½ hours, while on the weekends, the mice had approximately 4½ hours of access to chow. Mice were treated daily (weekdays and weekends).

Mice were treated daily with fluasterone, for about one month. Then the mice were sacrificed with an overdose of CO₂. The seminal vesicles of each mouse were excised, cleaned of adventitia and the coagulating glands, and any liquid was pressed out and then the glands were weighed on a Mettler AE50 balance.

Fasting Plasma Glucose Determinations

Chow was removed from the mice at approximately 3:30 p.m the day before drug treatment. This was done so that a fasting plasma glucose level could be obtained on the next day. The day of drug treatment a pretreatment plasma glucose determination was made. The mice were lightly anesthetized with Isoflurane and were bled from the orbital sinus (400 μL of blood was taken). The blood was obtained between 10:00 a.m. and noon. Blood was kept on ice until analysis. After the blood for the glucose measurement was taken out, the remaining blood was centrifuged at 3000×g for 15 minutes. The plasma was removed and frozen for use in determining plasma triglyceride levels.

Plasma glucose levels were assayed by a Sigrna kit 510. Briefly, 0.2 mL of blood was added to 1.8 mL of distilled water. The contents of the test tube were mixed and 1.0 mL of 0.3 N barium hydroxide solution was added and the tube contents were mixed by swirling. One mL of 5% zinc sulfate solution was added to the test tube and mixed by shaking. The test tubes were centrifuged at 3000×g for 20 minutes. One-half mL of the clear supernatant was transferred to a clean test tube and 5 mL of Combined Enzyme-Color Reagent solution was added and mixed well. Blanks were made using 0.2 mL of distilled water instead of blood and standards containing 100, 200, and 300 mg/dL of glucose were also simultaneously assayed. The tubes were incubated at room temperature for 45 minutes and were read at 450 nm in a Beckman DU 640 spectrophotometer. All readings were completed within a 30 minute period after the end of the incubation period.

Triglyceride Assay

Plasma triglyceride levels were determined with a Sigma kit (334-UV ). The determination is based on the enzymatic hydrolysis of triglycerides to glycerol and free fatty acids by lipase. Glycerol is subsequently phosphorylated by ATP to produce glycerol-1-phosphate and ADP. ATP is regenerated by a pyruvate kinase-catalyzed reaction between ADP and phosphoenol pyruvate. The pyruvate formed in this reaction is subsequently reduced to lactate with simultaneous oxidation of equimolar amounts of NADH in the presence of lactate dehydrogenase. NADH absorbs at 340 nm, thus the decrease in absorbance, measured at this wavelength, is directly proportional to the triglyceride concentration in the sample.

Triglyceride Reagent A (containing ATP, lactate dehydrogenase, lipase, NADH, phosphoenol pyruvate, pyruvate kinase) and Triglyceride Reagent B were reconstituted with 10 mL and 2 mL of distilled water respectively. The Sample Regent is prepared by adding 0.25 mL of Triglyceride Reagent B to 10 mL of Triglyceride Reagent A. Blank, standards (50, 100 and 200 mg/dL) and sample tubes were prepared. One mL of Sample Reagent is added to all tubes. To the Blank tube, 20 μL of distilled water is added. For each Sample tube, 20 μL of plasma is added to the tube. All tubes were incubated at room temperature for 10 minutes. The tubes were read against a Reference containing distilled water. Triglyceride concentration in the samples were calculated by subtracting the absorbance of the Sample from the absorbance of the Blank and using the following formula:

Serum triglyceride concentration=ΔA

The results are tabulated hereinbelow:

Seminal Vesicle Weights

| Group | Body Weight gm | Seminal Vesicles mg | s.v./b.w. |
|---|---|---|---|
| Control, s.c. | 52.2 | 50.4 | 0.9655 |
| | 50.6 | 51.7 | 1.0217 |
| | 42.9 | 40.8 | 0.9510 |
| Average | 48.6 | 47.6 | 0.9794 |
| ±S.D. | ±5.0 | ±6.0 | ±0.0373 |
| 5 mg/kg, s.c. | 44.8 | 42.2 | 0.9420 |
| | 43.8 | 41.9 | 0.9566 |
| | 47.5 | 40.7 | 0.8568 |
| | 38.2 | 43.4 | 1.1361 |
| | 46.5 | 44.5 | 0.9570 |
| Average | 44.2 | 42.5 | 0.9697 n.s. |
| ±S.D. | ±3.6 | ±1.5 | ±0.1019 |
| 20 mg/kg, s.c. | 45.6 | 39.6 | 0.8684 |
| | 35.8 | 35.8 | 1.0000 |
| | 39.9 | 35.4 | 0.8872 |
| | 42.9 | 34.6 | 0.8065 |
| | — | | |
| Average | 41.1 | 36.4 n.s. | 0.8905 n.s. |
| ±S.D. | ±4.2 | ±2.2 | ±0.0807 |
| 5 mg/kg, i.p. | 40.6 | 85.6 | 2.1084 |
| | 50.6 | 81.8 | 1.6166 |
| | 46.0 | 71.9 | 1.5630 |
| | 39.9 | 81.0 | 2.0301 |
| | — | | |
| Average | 44.3 | 80.1 p < 0.005 | 1.8295 |
| | | | p < 0.01 |
| ±S.D. | ±5.0 | ±5.8 | ±0.2795 |

Administration of fluasterone by the i.p. route produced a highly significant increase in seminal vesicle weights whereas administration by the s.c. route at much higher doses had no apparent effect on seminal vesicle weights.

The effect of oral, subcutaneous and intraperitoneal administration of 16α-fluoro-5-androsten-17-one on fasting plasma glucose and triglyceride levels is tabulated below.

Effect of Oral, Subcutaneous, or Intraperitoneal Administration
of Fluasterone on Fasting Plasma Glucose Levels
Fasting Plasma Glucose Levels (mg/dL)

| Treatment | Control | 200 mg/kg | Control | 5 mg/kg | 5 mg/kg |
|---|---|---|---|---|---|
| days | oral | oral | s.c. | s.c. | i.p. |
| 0 | 254 ± 98.9 | 245 ± 72.1 | 245 ± 76.4 | 245 ± 102 | 270 ± 81.9 |
| | (N = 5) | (N = 5) | (N = 5) | (N = 5) | (N = 5) |
| 7 | 260 ± 86.0 | 181 ± 69.3 | 258 ± 40.6 | 173 ± 58.3 | 434 ± 264 |
| | (N = 5) | (N = 5) | (N = 5) | (N = 5) | (N = 5) |
| 14 | 458 ± 94.0 | 107 ± 12.2² | 487 ± 53.3 | 134 ± 29.8¹ | 410 ± 32.1 |
| | (N = 5) | (N = 5) | (N = 4) | (N = 5) | (N = 5) |
| 17 | 551 ± 91.7 | 104 ± 24.8² | 376 ± 11.3 | 96.0 ± 18.5¹ | 635 ± 77.3³ |
| | (N = 5) | (N = 5) | (N = 3) | (N = 5) | (N = 4) |

Mice were treated with fluasterone and plasma glucose levels determined as described.
¹Significantly less than s.c. Control, p < 0.001.
²Significantly less than oral Control, p < 0.001.
³Significantly greater than s.c. Control, p < 0.01.

As shown, administration of fluasterone at 200 mg/kg p.o. or 5 mg/kg s.c. to diabetic mice (BKS.Cg-m⁺/₊Lepr^{db} mice) reduced fasting plasma glucose (FPG) levels, whereas 5 mg/kg i.p. fluasterone did not reduce FPG levels.

Mortality

1 mouse from Control, s.c. group died about a week later.

1 mouse from 5 mg/kg, i.p. group died about two weeks later.

1 mouse from 200 mg/kg oral group and 1 mouse from Control, s.c. group died about 17 days later.

The effect on plasma triglyceride levels is tabulated hereinbelow.

Effect of Oral, Subcutaneous, or Intraperitoneal Administration
of Fluasterone on Fasting Plasma Triglyceride Levels
Fasting Plasma Triglyceride Levels (mg/dL)

| Treatment | Control | 200 mg/kg | Control | 5 mg/kg | 5 mg/kg |
|---|---|---|---|---|---|
| days | oral | oral | s.c. | s.c. | i.p. |
| 0 | 130 ± 38.7 | 126 ± 18.6 | 128 ± 25.3 | 126 ± 20.9 | 136 ± 38.4 |
| | (N = 5) | (N = 5) | (N = 5) | (N = 5) | (N = 5) |
| 7 | 188 ± 21.8 | 107 ± 38.9¹ | 173 ± 21.0 | 115 ± 33.3² | 91.1 ± 14.5² |
| | (N = 5) | (N = 5) | (N = 5) | (N = 5) | (N = 5) |
| 14 | 161 ± 17.3 | 95.7 ± 15.6³ | 156 ± 21.6 | 115 ± 15.7⁴ | 115 ± 14.2⁴ |
| | (N = 5) | (N = 5) | (N = 4) | (N = 5) | (N = 5) |
| 17 | 161.6 ± 19.6 | 100 ± 13.1³ | 146 ± 19.5 | 115 ± 13.7 | 116 ± 17.0 |
| | (N = 5) | (N = 5) | (N = 3) | (N = 5) | (N = 4) |

Mice were treated with fluasterone and plasma triglyceride levels were determined as described on Pages 3 to 5.
¹Significantly less than oral Control, p < 0.01.
²Significantly less than s.c. Control, p < 0.02.
³Significantly less than oral Control, p < 0.001.
⁴Significantly less than s.c. Control, p < 0.01.

All three doses, however, significantly reduced fasting plasma triglyceride levels. Without wishing to be bound, it is believed that the androgenicity of i.p. administered fluasterone masked its anti-hyperglycemic effect.

Without wishing to be bound, it is believed that drugs injected i.p. enter the circulation rapidly primarily by way of the portal vein. The androgenicity of i.p. administered fluasterone, and the lack of androgenicity of s.c. administration, indicates that fluasterone is metabolized into androgenic species in the liver. The lack of androgenicity of the 200 mg/kg p.o. dose suggests that in the mouse orally administered fluasterone is absorbed primarily with fat droplets into the lymphatic system and bypasses first pass hepatic metabolism.

Since buccal or s.c. administered fluasterone does not undergo first pass liver metabolism the buccal or s.c. formulations of fluasterone will be more effective as anti-diabetic agents than orally administered formulations. Thus buccal or s.c. formulations of fluasterone are advantageous to oral formulations not only in having reduced androgenic side-effect, such as HDL lowering, but also in producing greater anti-diabetic efficacy.

The above preferred embodiments and examples are given to illustrate the scope and spirit of the present invention. The embodiments and examples described herein will make apparent to those skilled in the art other embodiments and examples. These other embodiments and examples are within the contemplation of the present invention. Therefore, the present invention should be limited only by the appended claims.

What is claimed is:

1. A method for treating a patient having hypertriglyceridemia comprising administering thereto a therapeutically effective amount of a compound of the formula: 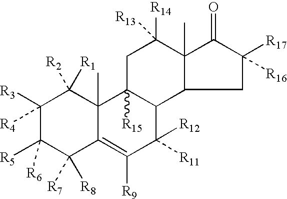
wherein:
R₁, R₂, R₃, R₄, R₇, R₈, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₉ is hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

2. The method according to claim 1 wherein R₁₆ and R₁₇ are independently hydrogen, lower alkyl, hydroxy, loweralkoxy or halogen.

3. The method according to claim 2 wherein R₁₇ is hydrogen and R₁₆ is lower alkyl, hydroxy, lower alkoxy or halogen.

4. The method according to claim 2 wherein R₁₆ and R₁₇ are independently hydrogen, fluoro or chloro.

5. The method according to claim 2 wherein R₁₆ is fluoro and R₁₇ is hydrogen.

6. The method according to claim 1 wherein R₅ and R₆ are hydrogen.

7. The method according to claim 2 wherein R₅ and R₆ are hydrogen.

8. The method according to claim 3 wherein R₅ and R₆ are hydrogen.

9. The method according to claim 8 wherein R₁₆ is fluoro.

10. The method according to claim 1 wherein R₁, R₂, R₃, R₄, R₇, R₈, R₁₃, R₁₄ and R₁₅ are hydrogen, R₉ is hydrogen, lower alkyl, or halogen and R₁₁ and R₁₂ are independently hydrogen, hydroxy, lower alkoxy, halogen or lower alkyl and R₁₆ and R₁₇ are independently hydrogen, halogen, lower alkyl, lower alkoxy or hydroxy and R₅ and R₆ are independently hydrogen or lower alkyl.

11. The method according to claim 10 wherein R₁₆ is fluoro, chloro, methyl, methoxy, R₁₇ is hydrogen, R₉ is hydrogen, methyl or fluoro or chloro and R₁₁ and R₁₂ are independently hydrogen, hydroxy, methoxy or methyl, fluoro or chloro.

12. The method according to claim 11 wherein R₁₆ is fluoro.

13. The method according to claim 10 wherein one of R₉, R₁₁ and R₁₂ is other than hydrogen.

14. The method according to claim 10 wherein R₅ and R₆ are hydrogen.

15. The method according to claim 14 wherein R₁₇ is hydrogen or halogen; R₁₁ and R₁₂ are independently hydrogen, hydroxy, lower alkyl, R₉ is hydrogen.

16. The method according to claim 14 wherein R₁₇ is hydrogen; R₉ is hydrogen; R₁₁ and R₁₂ are independently hydrogen or hydroxy and R₁₆ is fluoro, chloro or methyl.

17. The method according to claim 10 wherein R₁₆ is chloro or fluoro.

18. The method according to claim 1 wherein the compound is 16α-fluoro-5-androsten-17-one or 16α-fluoro-7β-hydroxy-5-androsten-17-one.

19. The method according to claim 1 or 10 wherein the patient has in addition at least one of the following characteristics:
(a) is insulin resistant
(b) is obese
(c) has a HDL level less than about 40 mg/dl if male and less than about 45 mg/dl if female.

20. The method according to claim 19 wherein the patient has a BMI greater than about 30 Kg/m².

21. A method for treating a patient having hypertriglyceridemia comprising administering thereto a therapeutically effective amount of a compound of the formula: 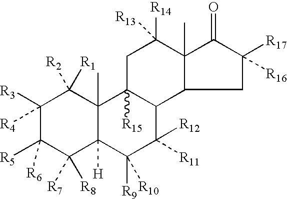
wherein
R₁, R₂, R₃, R₄, R₇, R₈, R₉, R₁₀, R₁₁, R₁₂, R₁₃, R₁₄ and R₁₅ are independently hydrogen, alkyl, halogen, hydroxy or alkoxy;
R₅ and R₆ are independently hydrogen, hydroxy, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

22. The method according to claim 21 wherein R₅ or R₆ is other than hydroxy.

23. The method according to claim 21 wherein R₁₆ and R₁₇ are independently hydrogen, lower alkyl, hydroxy, loweralkoxy or halogen.

24. The method according to claim 23 wherein R₁₇ is hydrogen and R₁₆ is lower alkyl, hydroxy, lower alkoxy or halogen.

25. The method according to claim 23 wherein R₁₆ and R₁₇ are independently hydrogen, fluoro or chloro.

26. The method according to claim 25 wherein R₁₆ is fluoro and R₁₇ is hydrogen.

27. The method according to claim 21 wherein R₅ and R₆ are hydrogen.

28. The method according to claim 23 wherein R₅ and R₆ are hydrogen.

29. The method according to claim 24 wherein R₅ and R₆ are hydrogen.

30. The method according to claim 29 wherein R₁₆ is fluoro.

31. The method according to claim 21 wherein R₁, R₂, R₃, R₄, R₇, R₈, R₁₃, R₁₄ and R₁₅ are hydrogen, R₉ is hydrogen, lower alkyl, or halogen and R₁₁ and R₁₂ are independently hydrogen, hydroxy, lower alkoxy, halogen or lower alkyl, R₁₆ and R₁₇ are independently hydrogen, halogen, lower alkyl, lower alkoxy or hydroxy and R₅ and R₆ are independently hydrogen or lower alkyl.

32. The method according to claim 31 wherein R₁₆ is fluoro or chloro, methyl, methoxy, R₁₇ is hydrogen, R₉ is hydrogen, methyl or fluoro or chloro and R₁₁ and R₁₂ are independently hydrogen, hydroxy, methoxy or methyl, fluoro or chloro, and R₅ and R₆ are hydrogen.

33. The method according to claim 31 wherein R₁₆ is fluoro.

34. The method according to claim 21 wherein one of R₉, R₁₁ and R₁₂ is other than hydrogen, R₁₇ is hydrogen and R₁₆ is chloro or fluoro.

35. The method according to claim 21 wherein R₅ and R₆ are hydrogen.

36. The method according to claim 35 wherein R₁₇ is hydrogen or fluoro; R₁₁ and R₁₂ are independently hydrogen, hydroxy, lower alkyl or halogen; R₉ is hydrogen, and R₁₆ is fluoro or chloro.

37. The method according to claim 35 wherein R₁₇ is hydrogen; R₉ is hydrogen and R₁₁ and R₁₂ are independently hydrogen or hydroxy and R₁₆ is fluoro, chloro or methyl.

38. The method according to claim 37 wherein R₁₆ is chloro or fluoro.

39. The method according to claim 21 wherein the compound is 16α-fluoro-5α-androstan-17-one or 16α-fluoro-7β-hydroxy-5α-androsten-17-one.

40. The method according to claim 21 or 31 wherein the patient additionally has at least one of the following characteristics:
(a) is insulin resistant
(b) is obese
(c) has a HDL level less than about 40 mg/dl if male and less than about 45 mg/dl if female.

41. The method according to claim 40 wherein the patient has a BMI greater than about 30 Kg/m².

42. A method of preventing atherosclerosis, coronary heart disease or stroke comprising administering to a patient a therapeutically effective amount of a compound of the formula: 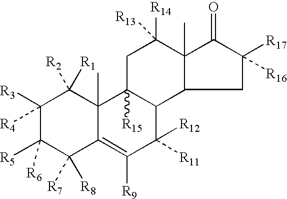
wherein
R₁, R₂, R₃, R₄, R₇, R₈, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₉ is hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

43. The method according to claim 42 wherein R₅ and R₆ are hydrogen and R₁₆ is lower alkyl, hydroxy, lower alkoxy, or halogen and R₁₇ is hydrogen, lower alkyl, hydroxy, lower alkoxy or halogen.

44. The method according to claim 43 wherein R₁, R₂, R₃, R₄, R₇, R₈, R₉, R₁₃, R₁₄ and R₁₅ are hydrogen, R₁₁ is hydrogen and R₁₂ is hydrogen or hydroxy.

45. The method according to claim 43 wherein halogen is fluoro or chloro.

46. The method according to claim 43 wherein R₁₆ is fluoro and R₁₇ is hydrogen.

47. The method according to claim 44 wherein R₁₆ is fluoro and R₁₇ is hydrogen.

48. A method of preventing atherosclerosis, coronary heart disease or stroke comprising administering to a patient a therapeutically effective amount of a compound of the formula: 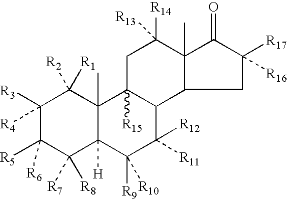
wherein
R₁, R₂, R₃, R₄, R₇, R₈, R₉, R₁₀, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

49. The method according to claim 48 wherein R₅ and R₆ are hydrogen and R₁₆ is lower alkyl, hydroxy, lower alkoxy, or halogen and R₁₇ is hydrogen, lower alkyl, hydroxy, lower alkoxy or halogen.

50. The method according to claim 49 wherein R₁, R₂, R₃, R₄, R₇, R₈, R₉, R₁₃, R₁₄ and R₁₅ are hydrogen, R₁₁ is hydrogen and R₁₂ is hydrogen or hydroxy.

51. The method according to claim 49 wherein halogen is fluoro or chloro.

52. The method according to claim 50 wherein R₁₆ is fluoro and R₁₇ is hydrogen.

53. A method of treating Syndrome X which comprises administering to a patient in need of treatment a therapeutically effective amount of a compound of the Formula: 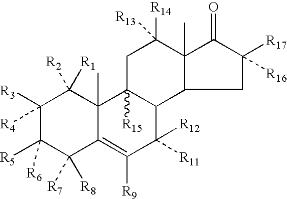
wherein
R₁, R₂, R₃, R₄, R₇, R₈, R₁₁, R₁₂, R₁₃, R₁₄ and R₁₅ are independently hydrogen, alkyl, halogen, hydroxy or alkoxy;
R₅ and R₆ are independently hydrogen, hydroxy alkyl, halogen or alkoxy;
R₉ is hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trilaloloweralkyl, with the proviso that only one of R₁₆ or R₁₇ may be alkenyl or alkynyl.

54. The method according to claim 53 wherein R₅ and R₆ are hydrogen and R₁₆ is lower alkyl, hydroxy, lower alkoxy, or halogen and R₁₇ is hydrogen, lower alkyl, hydroxy, lower alkoxy or halogen.

55. The method according to claim 54 wherein R₁, R₂, R₃, R₄, R₇, R₈, R₉, R₁₃, R₁₄ and R₁₅ are hydrogen, R₁₁ is hydrogen and R₁₂ is hydrogen or hydroxy.

56. The method according to claim 54 wherein halogen is fluoro or chloro.

57. The method according to claim 55 wherein R₁₆ is fluoro and R₁₇ is hydrogen.

58. A method of treating Syndrome X which comprises administering to a patient in need of treatment a therapeutically effective amount of a compound of the formula: 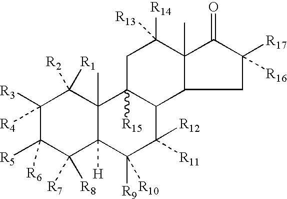
wherein
R₁, R₂, R₃, R₄, R₇, R₈, R₉, R₁₀, R₁₁, R₁₂, R₁₃, R₁₄ and R₁₅ are independently hydrogen, alkyl, halogen, hydroxy or alkoxy;
R₅ and R₆ are independently hydrogen, hydroxy, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

59. The method according to claim 58 wherein R₅ and R₆ are hydrogen and R₁₆ is lower alkyl, hydroxy, lower alkoxy, or halogen and R₁₇ is hydrogen, lower alkyl, hydroxy, lower alkoxy or halogen.

60. The method according to claim 59 wherein R₁, R₂, R₃, R₄, R₇, R₈, R₉, R₁₃, R₁₄ and R₁₅ are hydrogen, R₁₁ is hydrogen and R₁₂ is hydrogen or hydroxy.

61. The method according to claim 59 wherein halogen is fluoro or chloro.

62. The method according to claim 59 wherein R₁₆ is fluoro and R₁₇ is hydrogen.

63. The method according to claim 60 wherein R₁₆ is fluoro and R₁₇ is hydrogen.

64. The method according to any one of claims 1, 42 or 53 wherein the compound is 16α-fluoro-5-androsten-17-one, 7α-hydroxy-16α-fluoro-5-androsten-17-one or 7β-hydroxy-16α-fluoro-5-androsten-17-one.

65. The method according to any one of claims 21, 48 or 58 wherein the compound is 16α-fluoro-5α-androstan-17-one, 7α-hydroxy-16α-fluoro-5α-androstan-17-one or 16α-fluoro-7β-hydroxy-5α-androstan-17-one.

66. The method according to any one of claims 1, 21, 42, 48, 53 or 58 wherein the compound is administered buccally.

67. The method according to claim 64, where the compound is administered buccally.

68. The method according to claim 65 wherein the compound is administered buccally.

69. A method of lowering the concentration of excess glucocorticoid in a mammal which comprises administering to said mammal, an anti-glucocorticoid effective amount of a compound of the formula: 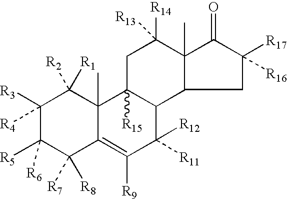
wherein
R₁, R₂, R₃, R₄, R₇, R₈, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₉ is hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

70. The method according to claim 69 wherein R₅ and R₆ are hydrogen and R₁₆ is lower alkyl, hydroxy, lower alkoxy, or halogen and R₁₇ is hydrogen, lower alkyl, hydroxy, lower alkoxy or halogen.

71. The method according to claim 70 wherein R₁, R₂, R₃, R₄, R₇, R₈, R₉, R₁₃, R₁₄ and R₁₅ are hydrogen, R₁₁ is hydrogen and R₁₂ is hydrogen or hydroxy.

72. The method according to claim 70 wherein halogen is fluoro or chloro.

73. The method according to claim 70 wherein R₁₆ is fluoro and R₁₇ is hydrogen.

74. The method according to claim 71 wherein R₁₆ is fluoro and R₁₇ is hydrogen.

75. The method according to claim 69 wherein the compound is 16α-fluoro-5-androsten-17-one, 7α-hydroxy-16α-fluoro-5-androsten-17-one or 7β-hydroxy-16α-fluoro-5-androsten-17-one.

76. The method according to claim 69 wherein the compound is administered buccally.

77. The method according to claim 75 wherein the compound is administered buccally.

78. The method of lowering the concentration of excess glucocorticoid in a mammal which comprises administering to said mammal an anti-glucocorticoid effective amount of a compound of the formula: 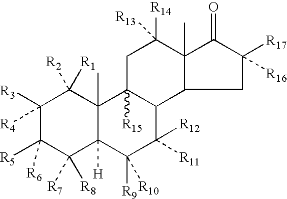
wherein
R₁, R₂, R₃, R₄, R₇, R₈, R₉, R₁₀, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

79. The method according to claim 78 wherein R₅ and R₆ are hydrogen and R₁₆ is lower alkyl, hydroxy, lower alkoxy, or halogen and R₁₇ is hydrogen, lower alkyl, hydroxy, lower alkoxy or halogen.

80. The method according to claim 79 wherein R₁, R₂, R₃, R₄, R₇, R₈, R₉, R₁₃, R₁₄ and R₁₅ are hydrogen, R₁₁ is hydrogen and R₁₂ is hydrogen or hydroxy.

81. The method according to claim 79 wherein halogen is fluoro or chloro.

82. The method according to claim 79 wherein R₁₆ is fluoro and R₁₇ is hydrogen.

83. The method according to claim 80 wherein R₁₆ is fluoro and R₁₇ is hydrogen.

84. The method according to claim 77 wherein the compound is 16α-fluoro-5α-androstan-17-one, 7α-hydroxy-16α-fluoro-5α-androstan-17-one or 7β-hydroxy-16α-fluoro-5α-androstan-17-one.

85. The method according to claim 77 wherein the compound is administered buccally.

86. The method according to claim 84 wherein the compound is administered buccally.

87. A method of minimizing the androgenicity of a steroid when administered to a patient, the steroid having the formula: 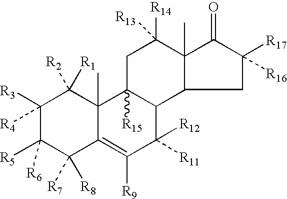
wherein:
R₁, R₂, R₃, R₄, R₇, R₈, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₉ is hydrogen, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl, said method comprising administering buccally said steroid to the patient.

88. The method according to claim 87 wherein R₅ and R₆ are hydrogen and R₁₆ is halo.

89. The method according to claim 88 wherein R₁₆ is chloro or fluoro.

90. The method according to claim 89 wherein R₁₆ is fluoro.

91. The method according to claim 87 wherein the compound is 16α-fluoro-5-androsten-17-one or 16α-fluoro-7β-hydroxy-5-androsten-17-one.

92. A method of minimizing the androgenicity of a steroid when administered to a patient, the steroid having the formula: 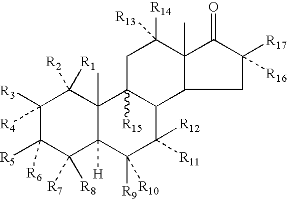
wherein
R₁, R₂, R₃, R₄, R₇, R₈, R₉, R₁₀, R₁₁, R₁₂, R₁₃, R₁₄ and R₁₅ are independently hydrogen, alkyl, halogen, hydroxy or alkoxy;
R₅ and R₆ are independently hydrogen, hydroxy, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl;
said method comprising administering said steroid to the patient buccally.

93. The method according to claim 92 wherein the steroid is 16α-fluoro-5α-androstan-17-one or 16α-fluoro-7β-hydroxy-5α-androsten-17-one.

94. The method according to claim 92 wherein R₅ and R₆ are hydrogen and R₁₆ is halo.

95. The method according to claim 94 wherein R₁₆ is chloro or fluoro.

96. The method according to claim 95 wherein R₁₆ is fluoro.

97. A method for treating or preventing a condition selected from the group consisting of hippocampal damage and immunosescence in a patient which comprises administering to said patient a therapeutically effective amount of a compound of the formula: 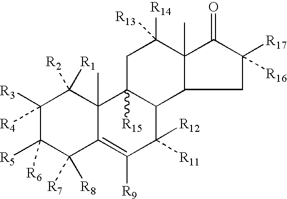
wherein:
R₁, R₂, R₃, R₄, R₇, R₈, R₁₁, R₁₂, R₁₃, R₁₄ and R₁₅ are independently hydrogen, alkyl, halogen, hydroxy or alkoxy;
R₅ and R₆ are independently hydrogen, alkyl, halogen or alkoxy;
R₉ is hydrogen, alkyl, halogen or alkoxy;
R₁₆ and R₁₇ are independently hydrogen, alkyl, halogen, hydroxy, lower alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

98. The method according to claim 97 wherein R₅ and R₆ are hydrogen and R₁₆ is halo.

99. The method according to claim 98 wherein R₁₆ is chloro or fluoro.

100. The method according to claim 99 wherein R₁₆ is fluoro.

101. The method according to claim 97 wherein the compound is 16α-fluoro-5-androsten-17-one or 16α-fluoro-7β-hydroxy-5-androsten-17-one.

102. A method for treating or preventing a condition selected from the group consisting of hippocampal damage and immunosescence in a patient which comprises administering to said patient a therapeutically effective amount of a compound of the formula: 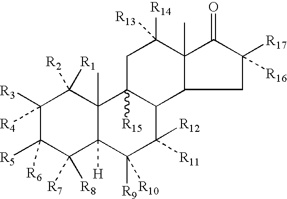
wherein
R₁, R₂, R₃, R₄, R₇, R₈, R₉, R₁₀, R₁₁, R₁₂, R₁₃, R₁₄ and R₁₅ are independently hydrogen, alkyl, halogen, hydroxy or alkoxy;
R₅ and R₆ are independently hydrogen, hydroxy, alkyl, halogen or alkoxy;
R₁₆ and R₁₇ are independently hydrogen, alkyl, halogen, hydroxy, alkoxy, lower alkenyl, lower alkynyl, amino, lower alkylamino, diloweralkylamino, lower alkoxy lower alkyl, hydroxy lower alkyl, amino lower alkyl, loweralkylamino lower alkyl, diloweralkylamino lower alkyl, haloloweralkyl, dihaloloweralkyl or trihaloloweralkyl, with the proviso that only one of R₁₆ or R₁₇ may be alkenyl or alkynyl.

103. The method according to claim 102 wherein R₅ and R₆ are hydrogen and R₁₆ is halo.

104. The method according to claim 103 wherein R₁₆ is chloro or fluoro.

105. The method according to claim 104 wherein R₁₆ is fluoro.

106. The method according to claim 102 wherein the steroid is 16α-fluoro-5α-androstan-17-one or 16α-fluoro-7β-hydroxy-5α-androstan-17-one.

107. The method according to any one of claims 1, 18, 21, 39, 42, 48, 53, 58, 69, 75, 78, 84, 87, 91, 92, 93, 97, 101, 102 or 106 wherein a statin is additionally present.
